SEQUENCE LISTING (30 sequences)

SEQ ID NO: 1 - DNA, 3357 bp, Clostridium histolyticum
  source 1..3357: mol_type = genomic DNA
atgaaaaaaaatattttaaagattcttatggatagttattctaaagaatctaaaattcaaactgtacgtagggttacgagtgtatcacttttagcggcatatcttactatgaatacttcaagtttagttttagcaaaaccaatagaaaatactaatgatactagtataaaaaatgtggagaaattaagaaatgctccaaatgaagagaatagtaaaaaggtagaagatagtaaaaatgataaggtagaacatgtggaaaatatagaagaggcaaaagttgagcaagttgcacccgaagtaaaatctaaatcaactttaagaagtgcttctatagcgaatactaattctgagaaatatgattttgagtatttaaatggtttgagctatactgaacttacaaatttaattaaaaatataaagtggaatcaaattaatggtttatttaattatagtacaggttctcaaaagttctttggagataaaaatcgtgtacaagctataattaatgctttacaagaaagtggaagaacttacactgcaaatgatatgaagggtatagaaactttcactgaggttttaagagctggtttttatttagggtactataatgatggtttatcttatttaaatgatagaaacttccaagataaatgtatacctgcaatgattgcaattcaaaaaaatcctaactttaagctaggaactgcagttcaagatgaagttataacttctttaggaaaactaataggaaatgcttctgctaatgctgaagtagttaataattgtgtaccagttctaaaacaatttagagaaaacttaaatcaatatgctcctgattacgttaaaggaacagctgtaaatgaattaattaaaggtattgaattcgatttttctggtgctgcatatgaaaaagatgttaagacaatgccttggtatggaaaaattgatccatttataaatgaacttaaggccttaggtctatatggaaatataacaagtgcaactgagtgggcatctgatgttggaatatactatttaagtaaattcggtctttactcaactaaccgaaatgacatagtacagtcacttgaaaaggctgtagatatgtataagtatggtaaaatagcctttgtagcaatggagagaataacttgggattatgatgggattggttctaatggtaaaaaggtggatcacgataagttcttagatgatgctgaaaaacattatctgccaaagacatatacttttgataatggaacctttattataagagcaggggagaaggtatccgaagaaaaaataaaaaggctatattgggcatcaagagaagtgaagtctcaattccatagagtagttggcaatgataaagctttagaggtgggaaatgccgatgatgttttaactatgaaaatatttaatagcccagaagaatataaatttaataccaatataaatggtgtaagcactgataatggtggtctatatatagaaccaagagggactttctacacttatgagagaacacctcaacaaagtatatttagtcttgaagaattgtttagacatgaatatactcactatttacaagcgagatatcttgtagatggtttatggggacaaggtccattttatgaaaaaaatagattaacttggtttgatgaaggtacagctgaattctttgcaggatctacccgtacatctggtgttttaccaagaaaatcaatattaggatatttggctaaggataaagtagatcatagatactcattaaagaagactcttaattcagggtatgatgacagtgattggatgttctataattatggatttgcagttgcacattatctatatgaaaaagatatgcctacatttattaagatgaataaagctatattgaatacagatgtgaaatcttatgatgaaataataaaaaaattaagtgatgatgcaaataaaaatacagaatatcaaaaccatattcaagagttagcagataaatatcaaggagcaggcatacctctagtatcagatgattacttaaaagatcatggatataagaaagcatctgaagtatattctgaaatttcaaaagctgcttctcttacaaacactagtgtaacagcagaaaaatctcaatattttaacacattcactttaagaggaacttatacaggtgaaacttctaaaggtgaatttaaagattgggatgaaatgagtaaaaaattagatggaactttggagtcccttgctaaaaattcttggagtggatacaaaactttaacagcatactttacgaattatagagttacaagcgataataaagttcaatatgatgtagttttccatggggttttaacagataatgcggatattagtaacaataaggctccaatagcaaaggtaactggaccaagcactggtgctgtaggaagaaatattgaatttagtggaaaagatagtaaagatgaagatggtaaaatagtatcatatgattgggattttggcgatggtgcaactagtagaggcaaaaattcagtacatgcttacaaaaaaacaggaacatataatgttacattaaaagtaactgacgataagggtgcaacagctacagaaagctttactatagaaataaagaacgaagatacaacaacacctataactaaagaaatggaacctaatgatgatataaaagaggctaatggtccaatagttgaaggtgttactgtaaaaggtgatttaaatggttctgatgatgctgataccttctattttgatgtaaaagaagatggtgatgttacaattgaacttccttattcagggtcatctaatttcacatggttagtttataaagagggagacgatcaaaaccatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 2 - DNA, 3066 bp, Clostridium histolyticum
  source 1..3066: mol_type = genomic DNA
atgaaaaggaaatgtttatctaaaaggcttatgttagctataacaatggctacaatatttacagtgaacagtacattaccaatttatgcagctgtagataaaaataatgcaacagcagctgtacaaaatgaaagtaagaggtatacagtatcatatttaaagactttaaattattatgacttagtagatttgcttgttaagactgaaattgagaatttaccagacctttttcagtatagttcagatgcaaaagagttctatggaaataaaactcgtatgagctttatcatggatgaaattggtagaagggcaccacagtatacagagatagatcataaaggtattcctactttagtagaagttgtaagagctggattttacttaggattccataacaaggaattgaatgaaataaataagaggtcttttaaagaaagggtaataccttctatattggcaattcaaaaaaatcctaattttaaactaggtactgaagttcaagataaaatagtatctgcaacaggacttttagctggtaatgaaacagcgcctccagaagttgtaaataattttacaccaataattcaagactgtatcaaaaatatggacagatatgctcttgatgatttaaagtcaaaagcattatttaatgttttagctgcacctacctatgatataactgagtatttaagagctactaaagaaaaaccagaaaacactccttggtatggtaaaatagatgggtttataaatgaacttaaaaagttagctctttatggaaaaataaatgataataactcttggataatagataatggtatatatcatatagcacctttagggaagttacatagcaataataaaataggaatagaaactttaacagaggttatgaagatatatccttatttaagtatgcaacatttacaatcagcagatcaaattgagcgtcattatgattcaaaagatgctgaaggaaataaaatacctttagataagtttaaaaaggaaggaaaagagaaatactgtccaaaaacttatacatttgatgatggaaaagtaataataaaagctggtgctagggtagaagaagaaaaagttaaaagactatactgggcatcaaaggaagttaactctcaattctttagggtatatggaatagacaaaccattagaagaaggtaatccagatgatatattaacaatggttatctacaacagtcctgaagaatataaacttaatagtgttctatacggatatgatactaataatggtggtatgtatatagagccagatggaactttcttcacatatgaaagaaaagctgaagaaagcacatacacattagaagaattatttagacatgaatatacacactatttacaaggaagatatgcagttcctggtcaatggggaagaacaaaactttatgacaatgatagattaacttggtatgaagaaggtggagcagaattatttgcaggttctactagaacttctggaatattaccaagaaagagtatagtatcaaatattcataatacaacaagaaataatagatataagctttcagacactgtacattctaaatatggtgctagttttgaattctataattatgcatgtatgtttatggattatatgtataataaagatatgggtatattaaataaactaaatgatcttgcaaaaaataatgatgttgatggatatgataattatattagagatttaagttctaatcatgctttaaatgataaatatcaagatcatatgcaggagcgcatagataattatgaaaatttaacagtgccttttgtagctgatgattatttagtaagacatgcttataagaaccctaatgaaatttattctgaaatatctgaagtagcaaaattaaaggatgctaagagtgaagttaagaaatcacaatattttagtacctttactttgagaggtagttacacaggtggagcatctaaggggaaattagaagatcaaaaagcaatgaataagtttatagatgattcacttaagaaattagatacgtattcttggagtgggtataaaactttaactgcttatttcactaattataaagttgactcttcaaatagagttacttatgatgtagtattccacggatatttaccaaacgaaggtgattccaaaaattcattaccttatggcaagatcaatggaacttacaagggaacagagaaagaaaaaatcaaattctctagtgaaggctctttcgatccagatggtaaaatagtttcttatgaatgggatttcggagatggtaataagagtaatgaggaaaatccagagcattcatatgacaaggtaggaacttatacagtgaaattaaaagttactgatgacaagggagaatcttcagtatctactactactgcagaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggacagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataa

SEQ ID NO: 3 - protein, 1118 aa, Clostridium histolyticum
MKKNILKILMDSYSKESKIQTVRRVTSVSLLAAYLTMNTSSLVLAKPIENTNDTSIKNVEKLRNAPNEENSKKVEDSKNDKVEHVENIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGEKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKTGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 4 - protein, 1021 aa, Clostridium histolyticum
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPIIQDCIKNMDRYALDDLKSKALFNVLAAPTYDITEYLRATKEKPENTPWYGKIDGFINELKKLALYGKINDNNSWIIDNGIYHIAPLGKLHSNNKIGIETLTEVMKIYPYLSMQHLQSADQIERHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPDGTFFTYERKAEESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNHALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 5 - protein, 1008 aa, Clostridium histolyticum
IANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGEKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKTGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 6 - protein, 991 aa, Clostridium histolyticum
AVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPIIQDCIKNMDRYALDDLKSKALFNVLAAPTYDITEYLRATKEKPENTPWYGKIDGFINELKKLALYGKINDNNSWIIDNGIYHIAPLGKLHSNNKIGIETLTEVMKIYPYLSMQHLQSADQIERHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPDGTFFTYERKAEESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNHALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 7 - protein, 443 aa, Clostridium septicum
MSKKSFAKKVICTSMIAIQCAAVVPHVQAYALTNLEEGGYANHNNASSIKIFGYEDNEDLKAKIIQDPEFIRNWANVAHSLGFGWCGGTANPNVGQGFEFKREVGAGGKVSYLLSARYNPNDPYASGYRAKDRLSMKISNVRFVIDNDSIKLGTPKVKKLAPLNSASFDLINESKTESKLSKTFNYTTSKTVSKTDNFKFGEKIGVKTSFKVGLEAIADSKVETSFEFNAEQGWSNTNSTTETKQESTTYTATVSPQTKKRLFLDVLGSQIDIPYEGKIYMEYDIELMGFLRYTGNAREDHTEDRPTVKLKFGKNGMSAEEHLKDLYSHKNINGYSEWDWKWVDEKFGYLFKNSYDALTSRKLGGIIKGSFTNINGTKIVIREGKEIPLPDKKRRGKRSVDSLDARLQNEGIRIENIETQDVPGFRLNSITYNDKKLILINNI

SEQ ID NO: 8 - protein, 419 aa, Clostridium histolyticum
MLKKSFFKKAICASLVVLQCLILVSPAQTLASTDLPTKGKTSIELFNYEDHMAHCLGFGWCFGTASKEIGEDFEFKRAEEEGKTVYYLSARYNQNDPYAKGYYRAHDRLVMKVSNARFFIDHDSLTLGKAKVISLDPLASSTLQVVNKSNSEAKTSLSFGYETTESTSKTDHVKFGEKIGIKSSFNVKVPFIGEKSIETNLEFNSEQGWSNTKTNSVTTKHTISHTTTTPAKSRKKVRLNVLNKKSDIPYEGKIYMEYDIEFFGFLRYTGNARKDHPTDRPSVSVKFGGKNNMSAVDHIIDLYKHKDINGYSEWDWNWIEENFYDRFSEYSSNVASQYFGGIISGVFTNVGGTDVKVEEGRERPLKNTSSTEQNVEVQNFKSSKSKEFRVGSLTYTTPNGEQTIYPEDVSSLNANNNEN

SEQ ID NO: 9 - protein, 548 aa, Bacillus proteolyticus
MKMKMKLASFGLAAGLAAQVFLPYNALASTEHVTWNQQFQTPQFISGDLLKVNGTSPEELVYQYVEKNENKFKFHENAKDTLQLKEKKNDNLGFTFMRFQQTYKGIPVFGAVVTSHVKDGTLTALSGTLIPNLDTKGSLKSGKKLSEKQARDIAEKDLVANVTKEVPEYEQGKDTEFVVYVNGDEASLAYVVNLNFLTPEPGNWLYIIDAVDGKILNKFNQLDAAKPGDVKSITGTSTVGVGRGVLGDQKNINTTYSTYYYLQDNTRGNGIFTYDAKYRTTLPGSLWADADNQFFASYDAPAVDAHYYAGVTYDYYKNVHNRLSYDGNNAAIRSSVHYSQGYNNAFWNGSQMVYGDGDGQTFIPLSGGIDVVAHELTHAVTDYTAGLIYQNESGAINEAISDIFGTLVEFYANKNPDWEIGEDVYTPGISGDSLRSMSDPAKYGDPDHYSKRYTGTQDNGGVHINSGIINKAAYLISQGGTHYGVSVVGIGRDKLGKIFYRALTQYLTPTSNFSQLRAAAVQSATDLYGSTSQEVASVKQAFDAVGVK

SEQ ID NO: 10 - protein, 532 aa, Clostridium histolyticum
MKKKFLSFIIISAISLNISSMTVGAKQVKEIKPPKDKESISVLKTDLEKTKNIKSNNKEGDDVTKVVKSALKEEGNLGDFKVDNKETDVKGKKHLRSQMFIDGIPVYGSQVIIHTNKDGQVYSVNGKVDKQPKAQSFKNRVRIKDDKAIKIAEDSLGKEIKKNKNYHSESKLYLYKVNGDLQPVYLVKISSTEPEASFWHMFVSAENGKIVDKYNALSCQATHAQVRGVNSSGEHKILNGMFENGRYFLADSTRPSNGYILTYDANNQEYGFPGSLFSNLTGIFDSDRQKAGVDAHHNLTQVYDYYKNVLNRDSFDGKGASIISSVHVGNNLNNAFWNGRQILFGDGDGVTFSNLAKCLEVTAHEFTHAVTQSTAGLEYRFQSGALNEAFSDILGIAVHSDPNDWEIGEDIYTPNVAGDALRSMSNPRLYRQPDHMKDYLYWDYSMDKGGVHYNSGIPNKAAYLMGKEVGKDSMAKIYYHALVNYLTPQSTFEDARNAVVSSAIDLHGENSKEHKLAIKSWADVGVGEEAVR

SEQ ID NO: 11 - protein, 204 aa, Bacillus proteolyticus
STEHVTWNQQFQTPQFISGDLLKVNGTSPEELVYQYVEKNENKFKFHENAKDTLQLKEKKNDNLGFTFMRFQQTYKGIPVFGAVVTSHVKDGTLTALSGTLIPNLDTKGSLKSGKKLSEKQARDIAEKDLVANVTKEVPEYEQGKDTEFVVYVNGDEASLAYVVNLNFLTPEPGNWLYIIDAVDGKILNKFNQLDAAKPGDVKS

SEQ ID NO: 12 - protein, 194 aa, Clostridium histolyticum
KPPKDKESISVLKTDLEKTKNIKSNNKEGDDVTKVVKSALKEEGNLGDFKVDNKETDVKGKKHLRSQMFIDGIPVYGSQVIIHTNKDGQVYSVNGKVDKQPKAQSFKNRVRIKDDKAIKIAEDSLGKEIKKNKNYHSESKLYLYKVNGDLQPVYLVKISSTEPEASFWHMFVSAENGKIVDKYNALSCQATHAQ

SEQ ID NO: 13 - protein, 316 aa, Bacillus proteolyticus
ITGTSTVGVGRGVLGDQKNINTTYSTYYYLQDNTRGNGIFTYDAKYRTTLPGSLWADADNQFFASYDAPAVDAHYYAGVTYDYYKNVHNRLSYDGNNAAIRSSVHYSQGYNNAFWNGSQMVYGDGDGQTFIPLSGGIDVVAHELTHAVTDYTAGLIYQNESGAINEAISDIFGTLVEFYANKNPDWEIGEDVYTPGISGDSLRSMSDPAKYGDPDHYSKRYTGTQDNGGVHINSGIINKAAYLISQGGTHYGVSVVGIGRDKLGKIFYRALTQYLTPTSNFSQLRAAAVQSATDLYGSTSQEVASVKQAFDAVGVK

SEQ ID NO: 14 - protein, 307 aa, Clostridium histolyticum
VRGVNSSGEHKILNGMFENGRYFLADSTRPSNGYILTYDANNQEYGFPGSLFSNLTGIFDSDRQKAGVDAHHNLTQVYDYYKNVLNRDSFDGKGASIISSVHVGNNLNNAFWNGRQILFGDGDGVTFSNLAKCLEVTAHEFTHAVTQSTAGLEYRFQSGALNEAFSDILGIAVHSDPNDWEIGEDIYTPNVAGDALRSMSNPRLYRQPDHMKDYLYWDYSMDKGGVHYNSGIPNKAAYLMGKEVGKDSMAKIYYHALVNYLTPQSTFEDARNAVVSSAIDLHGENSKEHKLAIKSWADVGVGEEAVR

SEQ ID NO: 15 - protein, 500 aa, Clostridium perfringens
MIRFKKTKLIASIAMALCLFSQPVISFSKDITDKNQSIDSGISSLSYNRNEVLASNGDKIESFVPKEGKKTGNKFIVVERQKRSLTTSPVDISIIDSVNDRTYPGALQLADKAFVENRPTILMVKRKPININIDLPGLKGENSIKVDDPTYGKVSGAIDELVSKWNEKYSSTHTLPARTQYSESMVYSKSQISSALNVNAKVLENSLGVDFNAVANNEKKVMILAYKQIFYTVSADLPKNPSDLFDDSVTFNDLKQKGVSNEAPPLMVSNVAYGRTIYVKLETTSSSKDVQAAFKALIKNTDIKNSQQYKDIYENSSFTAVVLGGDAQEHNKVVTKDFDEIRKVIKDNATFSTKNPAYPISYTSVFLKDNSVAAVHNKTDYIETTSTEYSKGKINLDHSGAYVAQFEVAWDEVSYDKEGNEVLTHKTWDGNYQDKTAHYSTVIPLEANARNIRIKARECTGLAWEWWRDVISEYDVPLTNNINVSIWGTTLYPGSSITYN

SEQ ID NO: 16 - protein, 520 aa, Clostridium histolyticum
MKITKKGLRSLSRLMLITMITGLTYNYHLGSSFNGNRVVLANPNTKTDNLIKNNSDEIDEKIYGLSYDPYKILSYNGEKVENFVPAECSENSGKFTVIKREKKNISDSTTDISIMDSINDRTYPGAIQLANRDLIENKPNLISCERKPITISVDLPGMGEDGKKVVNSPTYSSVNSAINYLLDTWNSKYSSKYTIPTRMNYSDTMVYSKSQLSTMFGCNFKTLSKSLNIDFDSIFKGEKKAMILSYKQIFYTVSVDGPNRPSDLFGYSVTSKSLALKGVNNDNPPAYVSNVAYGRTVYVKLETTSKSSKVKAAFKALVENQDISSNAEYKDIINQSSFTATVLGGGAQKHNKVVTKDFDVIRNIIKNNSVYSPQNPGYPISYTSTFLKDNKIATVNNRTEYIETTATEYDSGKIMLDHSGVYVAQFEVTWDEVSYDKQGNEIIEHKSWSGNNSDRTAHFNTELYLKGNARNISIKAKECTGLAWEWWRTVVDAKNLPLVKERKLSIWGTTLYPRYSMEEK

SEQ ID NO: 17 - protein, 526 aa, Clostridium histolyticum
MLRRKVSTLLMTALITTSFLNSKPVYANPVTKSKDNNLKEVQQVTSKSNKNKNQKVTIMYYCDADNNLEGSLLNDIEEMKTGYKDSPNLNLIALVDRSPRYSSDEKVLGEDFSDTRLYKIEHNKANRLDGKNEFPEISTTSKYEANMGDPEVLKKFIDYCKSNYEADKYVLIMANHGGGAREKSNPRLNRAICWDDSNLDKNGEADCLYMGEISDHLTEKQSVDLLAFDACLMGTAEVAYQYRPGNGGFSADTLVASSPVVWGPGFKYDKIFDRIKAGGGTNNEDDLTLGGKEQNFDPATITNEQLGALFVEEQRDSTHANGRYDQHLSFYDLKKAESVKRAIDNLAVNLSNENKKSEIEKLRGSGIHTDLMHYFDEYSEGEWVEYPYFDVYDLCEKINKSENFSSKTKDLASNAMNKLNEMIVYSFGDPSNNFKEGKNGLSIFLPNGDKKYSTYYTSTKIPHWTMQSWYNSIDTVKYGLNPYGKLSWCKDGQDPEINKVGNWFELLDSWFDKTNDVTGGVNHYQW

SEQ ID NO: 18 - protein, 526 aa, Clostridium histolyticum
MLRRKVSTLLMTALITTSFLNSKPVYANPVTKSKDNNLKEVQQVISKSNKNKNQKVTIMYYCDADNNLEGSLLNDIEEMKTGYKDSPNLNLIALVDRSPRYSSDEKVLGEDFSDTRLYKIELNKANRLDGKNEFPEISTTSKYEANMGDPEVLKKFIDYCKSNYEADKYVLIMANHGGGAREKSNPRLNRAICWDDSNLDKNGEADCLYMGEISDHLTEKQSVDLLAFDACLMGTAEVAYQYRPGNGGFSADTLVASSPVVWGPGFKYDKIFDRIKAGGGTNNEDDLTLGGKEQNFDPATITNEQLGALFVEEQRDSTHANGRYDQHLSFYDLKKAESVKRAIDNLAVNLSNENKKSEIEKLRGSGIHTDLMHYFDEYSEGEWVEYPYFDVYDLCEKINKSENFSSKTKDLASNAMNKLNEMIVYSFGDPSNNFKEGKNGLSTFLPNGDKKYSTYYTSTKIPHWTMQSWYNSIDTVKYGLNPYGKLSWCKDGQDPEINKVGNWFELLDSWFDKTNDVTGGVNHYQW

SEQ ID NO: 19 - protein, 1118 aa, Clostridium histolyticum
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 20 - protein, 1021 aa, Clostridium histolyticum
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 21 - DNA, 1263 bp, Clostridium histolyticum
  source 1..1263: mol_type = genomic DNA
atgttaaaaaaatctttttttaaaaaggcaatttgcgcatctttggtggtgctacaatgtttgatattagtgtcaccagctcaaacattggcatcaacagatttgccgacaaaaggaaaaacttcaattgaactatttaactatgaagatcattaaatggctcattgtttgggatttggatggtgcttcggtacagcatcaaaagaaataggggaagattttgaatttaaaagagcagaagaagaaggaaaaacagtatattatttatcagctagatacaatcaaaatgatccttacgctaaaggctattatcgcgcgcatgataggcttgttatgaaggttagtaatgctaggttttttatcgatcatgattcattaactttaggaaaagctaaagttataagtctagatccactggcatcatcaactcttcaagtagtaaataaaagtaattctgaagctaaaacatcattatcttttggatatgaaactactgaaagtacttccaaaacggatcacgttaaattcggagaaaaaattggaattaagtcatcatttaatgttaaagttccatttataggagaaaaatcaattgaaacaaatcttgaattcaattcagagcagggttggtccaatacgaaaactaactctgtaactactaaacatacaatttctcatacaacaacaacacctgcaaagagcaggaaaaaggtacgattaaatgttcttaataaaaagtccgacataccatatgagggtaagatatatatggaatatgatatagagttttttggttttttaagatatactggaaatgcgcgtaaagatcatcctacagatagacctagtgtatcagtaaaatttgggggaaaaaataatatgagtgcggtagatcatattatagatttgtacaagcataaagatattaatggctattcagaatgggattggaattggattgaagaaaatttttatgatagatttagtgaatattcatctaatgttgctagtcaatattttgggggcattatttctggtgtatttactaatgtgggtggaacagatgtaaaagttgaagaaggtagagaaaggccacttaaaaatacaagttctacagaacaaaatgtcgaagtacagaattttaaaagctctaaatctaaagagtttagagtgggtagtttaacatatactactcctaatggagaacagaccatatatcctgaagacgtatcatctcttaacgctaacaacaatgagaattaa

SEQ ID NO: 22 - DNA, 1599 bp, Clostridium histolyticum
  source 1..1599: mol_type = genomic DNA
atgaaaaaaaaatttttaagttttattattatttctgccatatcacttaacatttcttctatgactgtgggggcaaagcaagtgaaagaaatcaaacctccaaaagataaagaatctatttctgtattaaaaacagatttagaaaaaaccaagaatataaaatctaataataaggagggggatgatgtaacaaaagtagttaagagtgctttaaaagaagaaggcaatttaggagattttaaggttgataataaagaaactgatgtaaaaggtaaaaagcacttgcgttcacaaatgtttatagatggtattcctgtatatggtagtcaagttataattcatactaataaagatggacaagtatatagcgtaaatggaaaagtagataaacagcctaaagctcaatcttttaagaaccgtgtaaggattaaggacgataaagctattaaaatagcagaagacagtttaggtaaggaaataaagaaaaacaaaaattatcattctgaaagtaagttgtacctatacaaggttaatggagatttacaacctgtgtatttggtaaagatatcatctacagaaccagaagcttcattttggcatatgtttgtaagtgctgaaaatggaaagatagttgataagtataatgctttatcatgccaagctacacatgctcaagtaagaggagttaatagcagtggagagcataaaatcttaaatggtatgtttgaaaatggaagatattttttagcagattcaacaagaccttcaaatggatatatattaacatatgatgctaataaccaagagtatggtttcccaggtagcttatttagtaatttaacaggcatttttgatagtgatagacaaaaggcaggagtagatgctcaccataatctaactcaagtatatgattattataaaaatgttttaaatagagatagttttgatggaaaaggtgctagtataatatcttctgtgcatgtaggaaataatttaaataatgctttctggaatggtagacaaatactttttggtgatggagacggagttacatttagtaacctagcaaaatgtttagaagttactgcccatgaatttacacatgcagttactcaaagtactgcaggtctagaatatagatttcaatctggtgctctaaatgaagctttttctgatattttaggtatagctgttcacagtgatccaaatgattgggaaattggagaagatatatacactcctaatgtagcaggagatgctttaagaagtatgtcaaatcctagattatatagacaaccagaccatatgaaggactatttatattgggattattcaatggataaaggtggagttcattataattcaggtattccaaataaagcagcttatttgatgggaaaagaagttggaaaagattcaatggctaaaatttattatcatgctttagtgaattatttaactcctcaaagtacatttgaagatgctagaaatgcagtagtatcatctgcaatagatttacatggtgagaatagtaaagaacataaacttgctataaaatcttgggcagatgtaggcgttggagaagaggcagtaagataa

SEQ ID NO: 23 - DNA, 1563 bp, Clostridium histolyticum
  source 1..1563: mol_type = genomic DNA
atgaagattacaaagaaaggcttaagatcattatcacgcttaatgttaattactatgataacaggattaacatacaattatcacctaggtagtagctttaatgggaatcgagtagtacttgcaaatccaaatacaaaaacagataatttaattaagaataatagtgatgaaatagacgaaaagatttatggattgtcttatgatccatataaaatattatcttataatggagaaaaggttgaaaactttgttccagctgaatgttccgagaattccggaaaatttactgtaataaaacgtgaaaagaaaaatatttcagattcaactacagatatttcaataatggattcaataaatgatagaacttatcctggtgctatacaactagcaaatagggatcttatagaaaataagcctaatttaatttcatgcgagagaaaacctattactataagtgttgatttacctggtatgggtgaggatgggaaaaaggttgttaattctccaacatactcttcagttaattcagcaataaattatttgctagatacatggaattcaaaatattcatctaaatatactatacctacaaggatgaattattctgatactatggtgtatagtaaatcacagttatctacaatgtttggatgtaactttaaaactttaagtaaatccttaaatatagattttgattctatatttaaaggcgaaaaaaaggctatgattctatcatataaacaaattttctacacagtgagtgtagatggacctaatcgcccatcagatttatttggttacagtgtaacttctaagagcttagctttaaaaggagtaaataatgataatcctccagcatacgtttccaatgttgcatatggtagaactgtttatgtaaaactagagacaacatctaagagttcaaaggttaaagcagcatttaaggcattagtagagaatcaagatataagtagtaatgcagaatataaagacataataaatcaaagttcatttacagctactgttctaggtggaggagcacaaaaacacaataaagtagttactaaagatttcgatgtaataagaaatattattaaaaataattcagtatatagcccacaaaatcctggatatcctatttcatatacaagtacatttttaaaagacaataaaatagcaactgtaaacaatagaacagaatatatagaaacaactgcaacagaatacgatagcggcaaaataatgcttgaccatagtggagtttatgttgctcaatttgaagtaacctgggatgaagttagttatgacaaacaaggaaatgaaataattgagcataaatcttggtctggaaacaatagtgatagaacagctcactttaatacagaactatatttaaaaggaaatgcaagaaacatttctataaaagcaaaagaatgtacaggccttgcttgggaatggtggagaactgttgtagatgctaaaaatttaccacttgtaaaagaaagaaagttatcaatatggggtacaacattatatcctagatattctatggaagagaaataa

SEQ ID NO: 24 - DNA, 1581 bp, Clostridium histolyticum
  source 1..1581: mol_type = genomic DNA
atgttaagaagaaaagtatcaacactattaatgacagctttgataactacttcatttttaaattccaaacccgtatatgcaaatccagtaactaaatccaaggataataacttaaaagaagtacaacaagttataagcaagagtaataaaaacaaaaatcaaaaagtaactattatgtactattgcgacgcagataataacttggaaggaagtctattaaatgatatcgaggaaatgaaaacaggatataaggatagtcctaatttaaatttaattgctcttgtagacagatccccaagatatagcagtgacgaaaaagttttaggtgaagattttagtgatacacgtctttataagattgaactcaataaggcaaatagattagacggtaaaaatgaatttccagaaataagtactactagtaaatatgaagctaacatgggggatcctgaagttcttaaaaaatttattgattattgtaaatctaattatgaggctgataaatatgtgcttataatggctaatcatggtggtggtgcaagggaaaaatcaaatccaagattaaatagagcaatttgctgggatgatagtaaccttgataaaaatggtgaagcagactgcctttatatgggtgaaatttcagatcatttaacagaaaaacaatcagttgatttacttgcctttgatgcatgccttatgggaactgcagaagtagcgtatcagtatagaccaggtaatggaggattttctgccgatactttagttgcttcaagcccagtagtttggggtcctggattcaaatatgataagattttcgataggataaaagctggtggaggaactaataatgaggatgatttaactttaggtggtaaagaacaaaactttgatcctgcaaccattaccaatgagcaattaggtgcattatttgtagaagagcaaagagactcaacacatgccaatggtcgctatgatcaacacttaagcttttatgatttaaagaaagctgaatcagtaaaaagagccatagataatttagctgttaatctaagtaatgaaaacaaaaaatctgaaattgaaaaattaagaggaagtggaattcatacagatttaatgcattacttcgatgaatattctgaaggagaatgggttgaatatccttattttgacgtgtatgatttatgtgaaaaaataaataaaagtgaaaattttagtagtaaaactaaagatttagcttcaaatgctatgaataaattaaatgaaatgatagtttattcttttggagaccctagtaataattttaaagaaggaaaaaatggattgagtacattcttacctaatggagataaaaaatattcaacttattatacatcaaccaagatacctcattggactatgcaaagttggtataattcaatagatacagttaaatatggattgaatccttacggaaaattaagttggtgtaaagatggacaagatcctgaaataaataaagttggaaattggtttgaacttctagattcttggtttgataaaactaatgatgtaactggaggagttaatcattaccaatggtaa

SEQ ID NO: 25 - protein, 6 aa, synthetic construct
  REGION 1..6: note = Description of Artificial Sequence: Synthetic 6xHis tag
HHHHHH

SEQ ID NO: 26 - protein, 12 aa, Clostridium septicum
DKKRRGKRSVDS

SEQ ID NO: 27 - protein, 11 aa, Clostridium histolyticum
NTSSTEQNVEV

SEQ ID NO: 28 - protein, 10 aa, Bacillus proteolyticus
AHELTHAVTD

SEQ ID NO: 29 - protein, 11 aa, Clostridium perfringens
ECTGLAWEWWR

SEQ ID NO: 30 - protein, 4 aa, Clostridium histolyticum
AVDK

(19) United States
(12) Patent Application Publication
(10) Pub. No.: US 2025/0222080 A1
(43) Pub. Date: Jul. 10, 2025

(54) CLOSTRIDIUM HISTOLYTICUM ENZYMES AND METHODS FOR THE USE THEREOF

(71) Applicant: Endo Biologics Limited, DUBLIN (IE)

(72) Inventor: Wayne K. HERBER, Coopersburg, PA (US)

(21) Appl. No.: 19/094,246

(22) Filed: Mar. 28, 2025

Related U.S. Application Data
(63) Continuation of application No. 18/927,301, filed on Oct. 25, 2024, now Pat. No. 12,263,209.
Application No. 18/927,301 is a continuation of application No. 18/615,520, filed on Mar. 25, 2024.
Application No. 18/615,520 is a continuation of application No. 16/816,097, filed on Mar. 11, 2020, now Pat. No. 11,975,054.
Application No. 16/816,097 is a continuation of application No. 15/669,286, filed on Aug. 4, 2017, now Pat. No. 10,603,365.
Application No. 15/669,286 is a division of application No. 14/328,772, filed on Jul. 11, 2014, now Pat. No. 9,757,435.
Application No. 14/328,772 is a continuation of application No. PCT/US13/20940, filed on Jan. 10, 2013.
(60) Provisional application No. 61/585,909, filed on Jan. 12, 2012.

(51) Int. Cl. A61K 38/48; A61K 38/16; C12N 9/50; C12N 9/52; C12Q 1/37; G01N 33/50; G01N 33/569

(52) U.S. Cl. CPC A61K 38/4886; C12N 9/50; C12N 9/52; C12Q 1/37; C12Y 304/24003; C12Y 304/24027; G01N 33/5044; G01N 33/56911; A61K 38/164; G01N 2333/96419; Y02A 50/30

(57) ABSTRACT

The invention relates to recombinant nucleic acid and polypeptides encoding collagenase I and collagenase II, methods for the preparation thereof and methods for the use thereof. The invention also encompasses methods related to releasing a composition comprising collagenase prior to therapeutic administration.

### RELATED APPLICATIONS

This application is a continuation of U.S. application Ser. No. 18/927,301, filed on Oct. 25, 2024, which is a continuation of U.S. application Ser. No. 18/615,520, filed on Mar. 25, 2024, which is a continuation of U.S. application Ser. No. 16/816,097 (now U.S. Pat. No. 11,975,054), filed on Mar. 11, 2020, which is a continuation of U.S. application Ser. No. 15/669,286 (now U.S. Pat. No. 10,603,365), filed on Aug. 4, 2017, which is a divisional of U.S. application Ser. No. 14/328,772 (now U.S. Pat. No. 9,757,435), filed on Jul. 11, 2014, which is a continuation of International Application No. PCT/US13/020940, filed on Jan. 10, 2013, which claims the benefit of U.S. Provisional Application No. 61/585,909, filed on Jan. 12, 2012, the disclosures of each of which are herein incorporated by reference in their entireties.

### SEQUENCE LISTING

The contents of the electronic sequence listing (117326.000608_Sequence Listing.xml; Size: 51,192 bytes; and Date of Creation: Mar. 22, 2024) is herein incorporated by reference in its entirety.

### BACKGROUND OF THE INVENTION

Collagen is the major structural constituent of mammalian organisms and makes up a large portion of the total protein content of skin and other parts of the animal body. In humans, it is particularly important in the processes of wound healing process and natural aging. Various skin traumas, including burns, surgery, and infection, are characterized by the accumulation of fibrous tissue rich in collagen and having increased proteoglycan content. In addition to the replacement of the normal tissue which has been damaged or destroyed, excessive and disfiguring deposits of new tissue sometimes form during the healing process. The excess collagen deposition has been attributed to a disturbance in the balance between collagen synthesis and collagen degradation.

Diseases and conditions associated with excess collagen deposition and the erratic accumulation of fibrous tissue rich in collagen can be referred to as “collagen-mediated diseases.” Collagenase, an enzyme that has the specific ability to digest collagen, has been used to treat a variety of collagen-mediated diseases, including, for example, Dupuytren's contracture, Peyronie's disease, lipoma and adhesive capsulitis. U.S. Pat. Nos. 6,086,872 and 5,589,171, incorporated herein by reference, disclose the use of collagenase preparations in the treatment of Dupuytren's disease. U.S. Pat. No. 6,022,539, incorporated herein by reference, discloses the use of collagenase preparations in the treatment of Peyronie's disease. U.S. Pat. Nos. 6,958,150 and 7,842,673, incorporated herein by reference, disclose the use of collagenase for the treatment of lipoma. U.S. Patent Application Publication No. 2006/020448A1, incorporated herein by reference, discloses the use of collagenase in the treatment of adhesive capsulitis. Collagenase for use in therapy may be obtained from a variety of sources including mammalian, fungal, and bacterial sources. One common source of crude collagenase is from a bacterial fermentation process, specifically the fermentation of Clostridium histolyticum (C. histolyticum). The crude collagenase obtained from C. histolyticum may be purified using any of a number of chromatographic techniques.

One drawback of the fermentation of bacteria is that various toxins will be produced, that if present in the therapeutic composition, would be detrimental to the health of the patient. For example, C. histolyticum fermentation results in the synthesis of the hemolytic toxins alpha and epsilon, which can cause lysis of red blood cells (hemolysis), potentially leading to hemolytic crisis and hemolytic anemia. Hemolytic crisis occurs when there is a rapid destruction of large numbers of red blood cells in conjunction with the body's inability to replenish the red blood cells quickly enough to reestablish normal red blood cell levels. A hemolytic crisis causes acute (and often severe) hemolytic anemia, and can result in fatigue, shortness of breath, dizziness, headache, coldness in the hands and feet, pale skin, chest pain, jaundice, pain in the upper abdomen, leg ulcers and pain, severe reactions to a blood transfusion, arrhythmias, an enlarged heart, and heart failure. In order to ensure that the therapeutic collagenase preparation does not contain hemolytic toxins that might be expressed during C. histolyticum fermentation, a method for releasing a drug product prior to administration to a patient is presented.

As discussed above, collagenase for use in therapy can be obtained from a variety of sources such as bacterial sources (e.g. from the fermentation of C. histolyticum). It would be useful to develop additional sources of collagenase such as recombinant forms of collagenase enzymes.

### SUMMARY OF THE INVENTION

In some aspects, the present invention is based on the discovery of mutated polynucleotide sequences that encode functional collagenase I and collagenase II. The invention thus encompasses recombinant nucleic acid and polypeptides comprising the novel polynucleotide or polypeptide sequences and methods for the use thereof. The present invention also provides a method for detecting the secretion of a hemolytic toxin by a bacterial production strain, wherein the production strain produces a collagenase, prior to therapeutic administration of said collagenase to a patient and methods for detecting the presence of a hemolytic toxin in a collagenase composition.

In one embodiment, the invention is directed to a recombinant nucleic acid molecule comprising a polynucleotide having the sequence of SEQ ID NO: 1 (collagenase I nucleotide sequence) or the complement of SEQ ID NO: 1. In certain aspects, the recombinant nucleic acid further comprises a heterologous regulatory sequence operably linked to the polynucleotide. In certain additional embodiments, the invention is a recombinant nucleic acid molecule consisting of a polynucleotide of SEQ ID NO: 1. In yet additional aspects, the invention relates to a recombinant nucleic acid molecule consisting of a polynucleotide of SEQ ID NO: 1 and a heterologous regulator sequence operably linked to the polynucleotide.

In another embodiment, the invention is a recombinant nucleic acid molecule comprising a polynucleotide having the sequence of SEQ ID NO: 2 (collagenase II nucleotide sequence) or the complement of SEQ ID NO: 2. In certain aspects, the recombinant nucleic acid further comprises a heterologous promoter operatively linked to the polynucleotide. In certain additional embodiments, the invention is a recombinant nucleic acid molecule consisting of a polynucleotide of SEQ ID NO: 2. In yet additional aspects, the invention relates to a recombinant nucleic acid molecule consisting of a polynucleotide of SEQ ID NO: 2 and a heterologous regulator sequence operably linked to the polynucleotide.

The invention also includes recombinant polypeptides encoded by a recombinant nucleic acid comprising a polynucleotide having the sequence of SEQ ID NO: 1 or SEQ ID NO: 2.

In certain additional embodiments, the invention is directed to an expression cassette comprising a recombinant nucleic acid, wherein the nucleic acid comprises a polynucleotide having the sequence of SEQ ID NO: 1 or SEQ ID NO: 2.

In yet an additional embodiment, the invention is directed to a vector comprising a recombinant nucleic acid, wherein the nucleic acid comprises a polynucleotide having the sequence of SEQ ID NO: 1 or SEQ ID NO: 2. In some embodiments, the vector is a plasmid.

In a further aspect, the invention is directed to a recombinant host cell comprising the vector or plasmid comprising a polynucleotide having the sequence of SEQ ID NO: 1 or SEQ ID NO: 2. The invention also encompasses a method of producing collagenase I or collagenase II comprising culturing the host cell under conditions suitable for expression of the nucleic acid and recovering the collagenase I or collagenase II. The invention also includes a collagenase enzyme produced by culturing the recombinant host cell.

In some embodiments, the invention is directed to a recombinantly produced collagenase I comprising the amino acid sequence of SEQ ID NO: 3, a recombinantly produced collagenase II comprising the amino acid sequence SEQ ID NO: 4, a recombinantly produced collagenase I comprising the amino acid sequence of SEQ ID NO: 5, or a recombinantly produced collagenase II comprising the amino acid sequence of SEQ ID NO: 6.

Also included in the present invention are pharmaceutical compositions comprising collagenase I as described herein, collagenase II as described herein, or a combination thereof. In certain aspects, the present invention is directed to a pharmaceutical composition comprising a pharmaceutically acceptable carrier and a polypeptide comprising the amino sequence of SEQ ID NO: 3, a polypeptide comprising the amino acid sequence of SEQ ID NO: 4, or a combination thereof. In certain additional aspects, the present invention is directed to a pharmaceutical composition comprising a pharmaceutically acceptable carrier and a polypeptide comprising the amino sequence of SEQ ID NO: 5, a polypeptide comprising the amino acid sequence of SEQ ID NO: 6, or a combination thereof. The invention additionally includes methods of treating a collagen-mediated disease comprising administering an effective amount of collagenase I, collagenase II, or a combination thereof.

As discussed above, the invention encompasses methods for detecting the secretion of a hemolytic toxin by a bacterial production strain and methods for detecting the presence of a hemolytic toxin in a collagenase composition.

In one embodiment of the invention, a bacterial strain that produces collagenase is tested for the production of hemolytic toxins using a hemolysis assay. In one aspect, the hemolysis assay is performed using a blood agar substrate.

In another embodiment, a collagenase product is tested for the presence of hemolytic toxins using a hemolysis assay. In certain aspects, the hemolysis assay is performed using a blood agar substrate. In additional aspects, the hemolysis assay is performed using photometric detection of released hemoglobin. The absence of hemolytic toxins, as determined by a hemolysis assay or photometric detection, would support the release of the drug product for therapeutic administration.

Various strains of collagenase-producing bacteria can be assayed for hemolytic activity according to a method of the invention, in support of the release of a collagenase drug product for therapeutic administration. For example, members of the genera Actinobacillus, Actinomadura, Bacillus, Bacteriodes, Bifidobacterium, Brucella, Capnocytophaga, Clostridium, Enterococcus, Escherichia, Eubacterium, Flavobacterium, Fusobacterium, Peptococcus, Peptostreptococcus, Porphyromonas, Prevotella, Proteus, Pseudomonas, Serratia, Staphylococcus, Streptomyces, Streptococcus, Treponema, and Vibrio can be assayed for hemolytic activity according to a method of the invention, in support of the release of a collagenase drug product for therapeutic administration.

In another embodiment, a collagenase product produced by, and purified from, a strain of collagenase-producing bacteria is assayed for hemolytic activity according to a method of the invention, in support of the release of a collagenase drug product for therapeutic administration. In some embodiments, the production strain is selected from, but not limited to, the above-listed genera. In another aspect of the invention, the production strain is an Escherichia coli (E. coli) strain, including forms of E. coli that have been transformed with recombinant forms of collagenase I and collagenase II. In certain aspects of the invention, the production strain is a Clostridium perfringens (C. prefrigens) strain. In additional aspects, the production strain is a C. histolyticum strain.

In yet another embodiment of the invention, the collagenase composition is assayed for hemolytic activity according to a method of the invention, wherein the collagenase composition comprises a combination of purified C. histolyticum collagenase I and collagenase II. In an additional embodiment, the invention is a method of producing a drug product consisting of C. histolyticum collagenase I and II, wherein said method comprises testing a bacterial production strain for the absence of a functional, secreted hemolytic toxin according to a method of the invention.

In yet another embodiment, the invention is a method of purifying a crude collagenase composition, wherein said method comprises purifying the composition by filtration and column chromatography, followed by confirming the absence of a hemolytic toxin according to a method described herein.

In a further embodiment, the invention is a method of treating a collagen-mediated condition in a patient in need thereof, wherein said method comprises administering to said patient an effective amount of a drug product comprising collagenase, wherein the absence of a hemolytic toxin in said drug product or in a bacterial production strain producing said collagenase is confirmed according to a method of the invention prior to administration of said drug product to a patient, and/or formulation of the collagenase in a pharmaceutical composition.

Kits for testing for the presence or absence of hemolytic toxins in a sample are also described.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows protein alignment of Clostridium septicum (C. septicum) alpha toxin with the putative alpha toxin of C. histolyticum CLH_2834 and 2835. The C. septicum alpha toxin amino acid sequence (SEQ ID NO: 7) is the upper sequence in each row. The C. histolyticum CLH_2834 & 2835 (SEQ ID NO: 8) is the lower sequence in each row. The underlined, shaded sequence is the N-terminus of the mature C. septicum alpha toxin. The asterisks above the amino acids shows non-conserved essential residues critical for functionality (identifies mismatch in sequence). The shading shows conserved essential residues (confirms identity). The sequence numbering is based on the C. septicum sequence.

FIG. 2 shows blood agar plating of C. septicum. The arrows indicate beta hemolytic activity.

FIG. 3 shows amino acid alignment of Bacillus proteolyticus thermolysin with the putative delta toxin of C. histolyticum CLH_2576. The upper sequence in each row shows the sequence of Bacillus proteolyticus (B. proteolyticus) thermolysin protein (SEQ ID NO: 9). The lower sequence in each row is the sequence of C. histolyticum CLH_2576 (SEQ ID NO: 10). The green shading shows the proprotein region. The numbering is based on the thermolysin sequence.

FIG. 4 shows the prosequence amino acid alignment of B. proteolyticus thermolysin with the putative delta toxin of C. histolyticum CLH_2576. The upper sequence in each row is the prosequence of B. proteolyticus thermolysin protein (SEQ ID NO: 11). The lower sequence in each row is the prosequence of C. histolyticum CLH_2576 (SEQ ID NO: 12). The asterisks above the amino acids show the non-conserved essential residues critical for functionality (identifies mismatch in sequence). The green shading shows the conserved essential residues (confirms identity). The numbering is based on the thermolysin sequence.

FIG. 5 shows the mature sequence protein alignment of B. proteolyticus thermolysin with the putative delta toxin of C. histolyticum CLH_2576. The upper sequence in each row is the mature sequence of B. proteolyticus thermolysin (SEQ ID NO: 13). The lower sequence in each row is C. histolyticum CLH_2576 (SEQ ID NO: 14). The asterisks above the amino acids show non-conserved essential residues critical for functionality (identifies mismatch in sequence). The green shading shows conserved essential residues (confirms identity). The numbering is based on thermolysin sequence.

FIG. 6 shows the protein alignment of C. perfringens perfringolysin with the putative epsilon toxin of C. histolyticum CLH_1920. The upper sequence in each row is C. perfringens perfringolysin amino acid sequence (SEQ ID NO: 15). The lower sequence in each row is the amino acid sequence of C. histolyticum CLH_1920 (SEQ ID NO: 16). The blue star shows the signal peptidase cleavage site of perfringolysin K43. The asterisks above the amino acids show non-conserved essential residues critical for functionality (identifies mismatch in sequence). The green shading shows conserved essential residues (confirms identity). The numbering is based on the perfringolysin sequence.

FIG. 7 shows the beta hemolytic phenotype of tetanolysin.

FIG. 8 shows the protein alignment of C. histolyticum clostripain with the putative gamma toxin of C. histolyticum CLH_1861. The upper sequence in each row is C. histolyticum clostripain amino acid sequence (SEQ ID NO: 17). The lower sequence in each row is C. histolyticum CLH_1920 amino acid sequence (SEQ ID NO: 18). The asterisks above the amino acids shows non-conserved essential residues critical for functionality (identifies mismatch in sequence). The green shading shows the conserved essential residues (confirms identity). The numbering based on clostripain X63673 sequence.

FIG. 9A and 9B show an alignment comparison of the translated amino acid sequence from colG and the amino acid sequence of SEQ ID NO: 3 (the translated amino acid sequence from CLH_1768 and 1769; the upper sequence). As shown in FIG. 9A and 9B, the mature protein encoded by the amino acid sequence of SEQ ID NO: 3 differs from the translated amino acid sequence from colG amino acid sequence by three amino acids. The N-terminus of the mature protein begins at Ile 119 of the sequence of SEQ ID NO: 3. The amino acid sequence of the mature protein beginning at Ile 119 of SEQ ID NO: 3 is SEQ ID NO: 5.

FIG. 10A and 10B show an alignment comparison of the translated amino acid sequence from colH and SEQ ID NO: 4 (the translated amino acid sequence from CLH_2116; the bottom sequence). As shown in FIG. 10A and 10B, the mature protein encoded by the amino acid sequence of SEQ ID NO: 4 differs from the translated colG amino acid sequence by eight amino acids. The N-terminus of the mature protein begins at Ala 31 in colG and in SEQ ID NO: 4. The amino acid sequence of the mature protein beginning at Ala 31 of SEQ ID NO: 4 is SEQ ID NO: 6.

FIG. 11A and 11B show the nucleotide sequence of SEQ ID NO: 1 (CLH_1768 and 1769; collagenase I).

FIG. 12A and 12B show the nucleotide sequence of SEQ ID NO: 2 (CLH_2116; collagenase II).

FIG. 13A and 13B show the amino acid and nucleotide sequence of SEQ ID NO: 8 and SEQ ID NO: 21, respectively (CLH_2835 and CLH_2834; alpha toxin).

FIG. 14A and 14B show the amino acid and nucleotide sequence of SEQ ID NO: 10 and SEQ ID NO: 22, respectively (CLH_2576; delta toxin).

FIG. 15A and 15B show the amino acid and nucleotide sequence of SEQ ID NO: 16 and SEQ ID NO: 23, respectively (CLH_1920; epsilon toxin).

FIG. 16A and 16B show the amino acid and nucleotide sequence of SEQ ID NO: 18 and SEQ ID NO: 24, respectively (CLH_1861; gamma toxin).

FIG. 17A shows the amino acid sequence of SEQ ID NO: 3 (colG).

FIG. 17B shows the amino acid sequence of SEQ ID NO: 5.

FIG. 18A shows the amino acid sequence of SEQ ID NO: 4 (colH).

FIG. 18B shows the amino acid sequence of SEQ ID NO: 6.

### DETAILED DESCRIPTION OF THE INVENTION

A description of preferred embodiments of the invention follows.

The words “a” or “an” are meant to encompass one or more, unless otherwise specified. For example, “a hemoloytic toxin” refers to one or more hemolytic toxins.

The practice of the present invention will employ, unless otherwise indicated, conventional techniques of cell culture, molecular biology, microbiology, cell biology, and immunology, which are well within the skill of the art. Such techniques are fully explained in the literature. See, e.g., Sambrook et al., 1989, “Molecular Cloning: A Laboratory Manual”, Cold Spring Harbor Laboratory Press; Ausubel et al. (1995), “Short Protocols in Molecular Biology”, John Wiley and Sons; Methods in Enzymology (several volumes); Methods in Cell Biology (several volumes), and Methods in Molecular Biology (several volumes); the contents of each of which are expressly incorporated by reference herein.

### A. Recombinant Nucleic Acids And Proteins

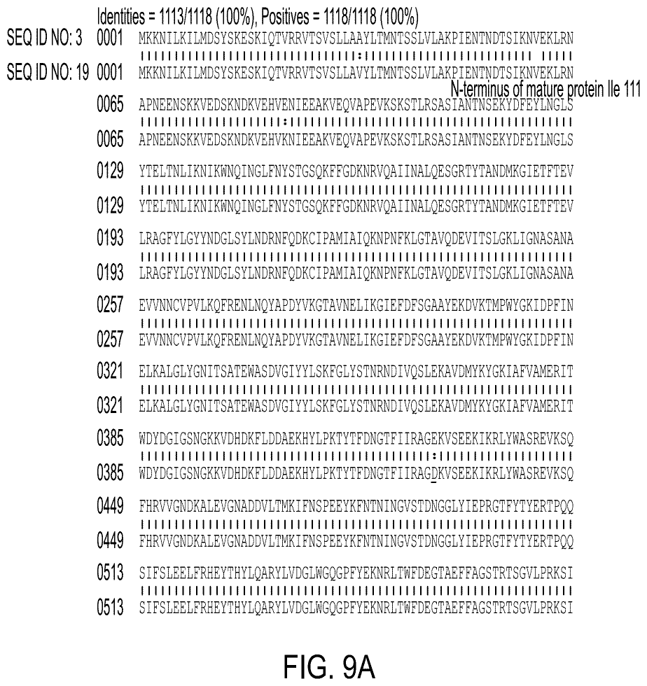

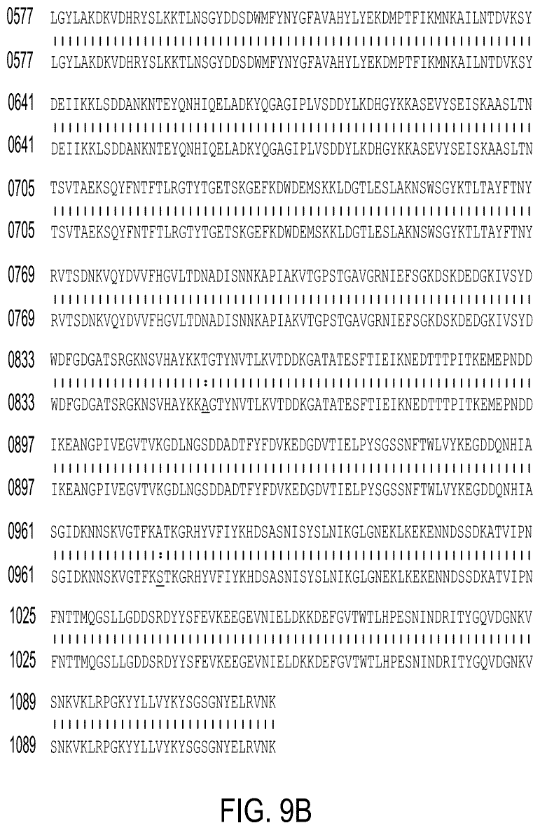

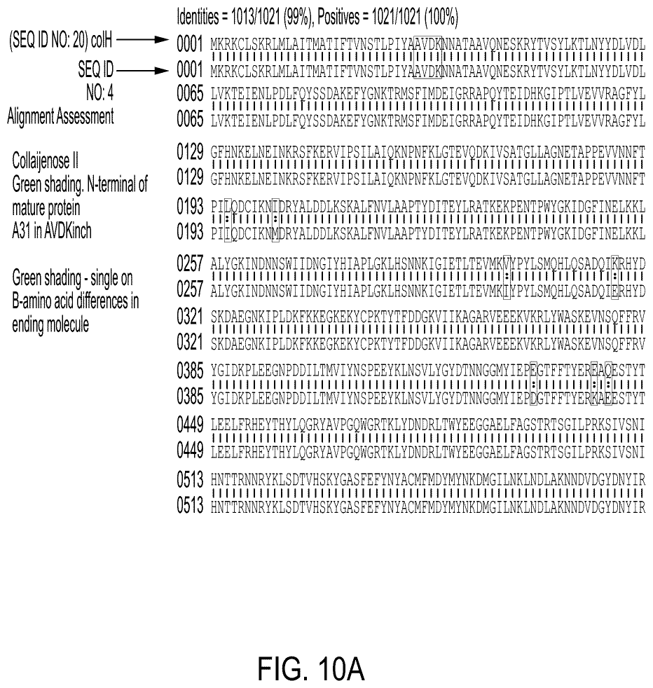

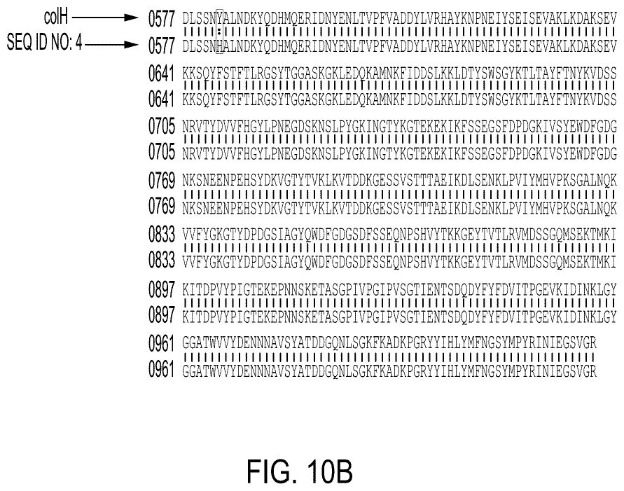

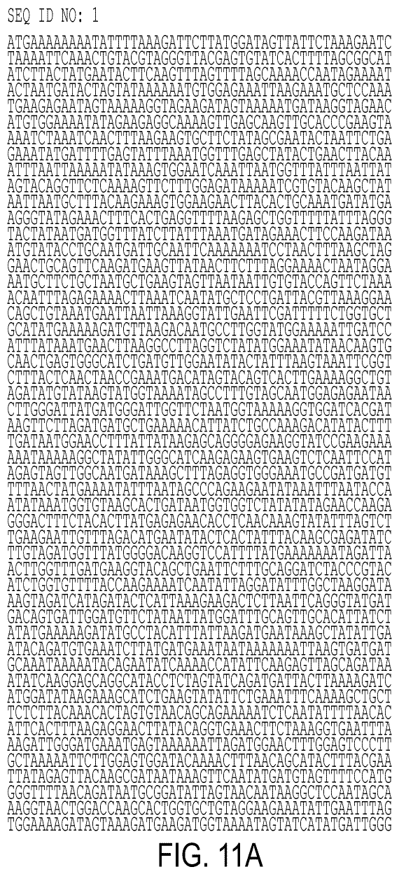

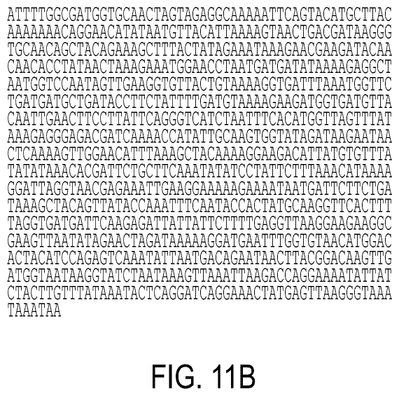

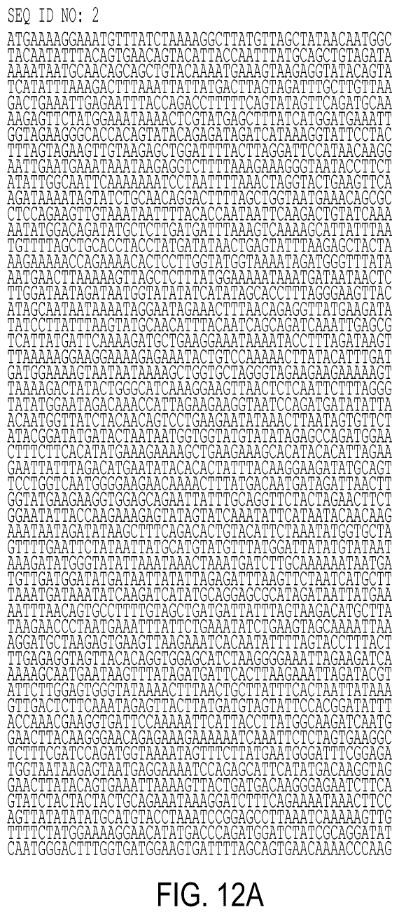

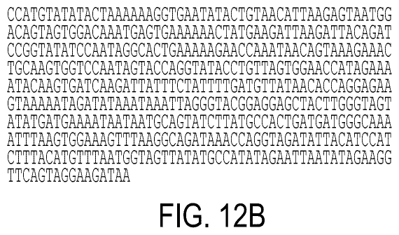

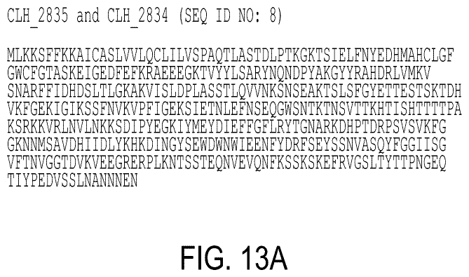

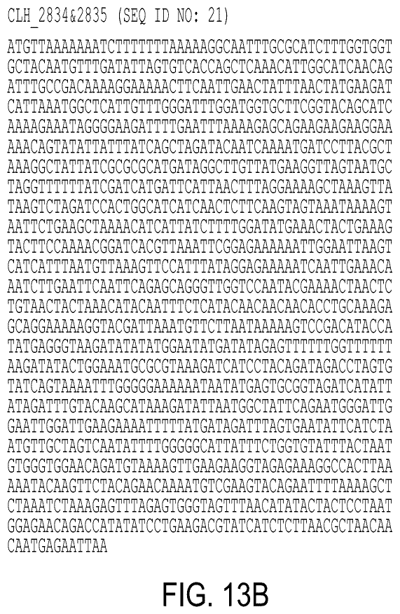

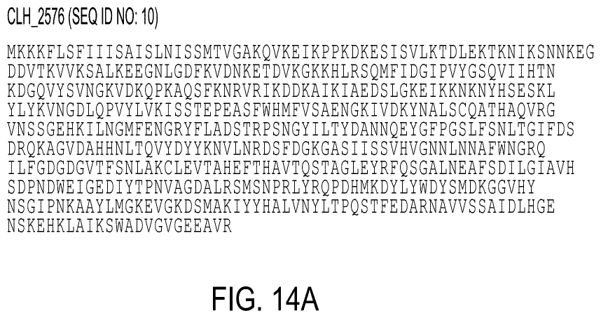

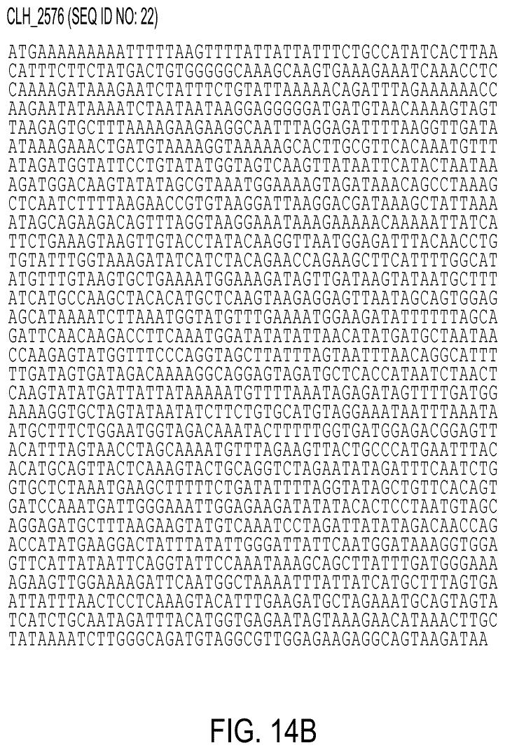

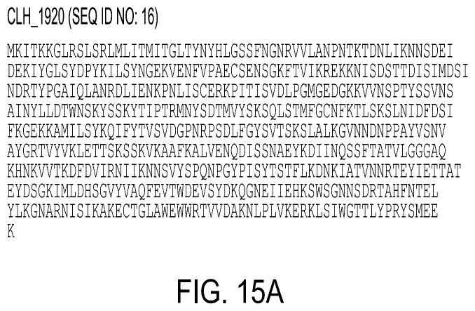

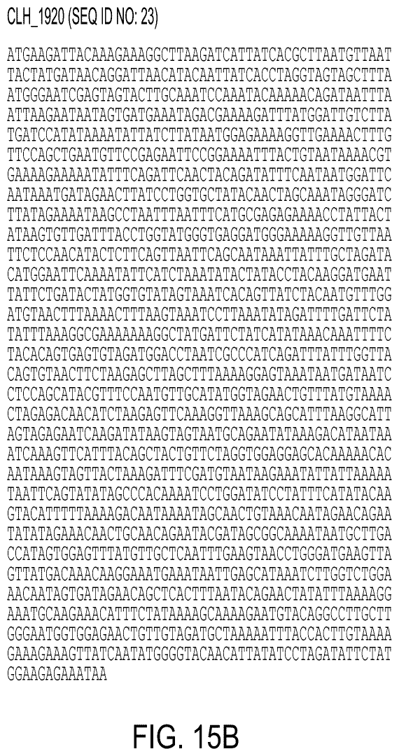

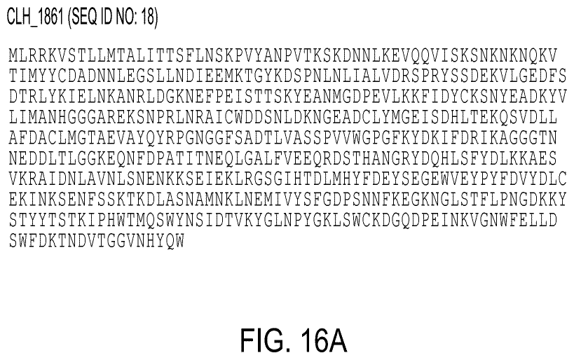

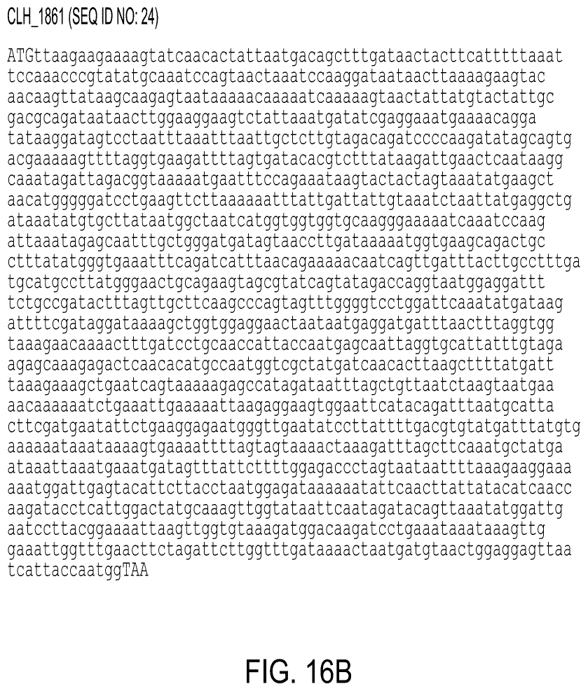

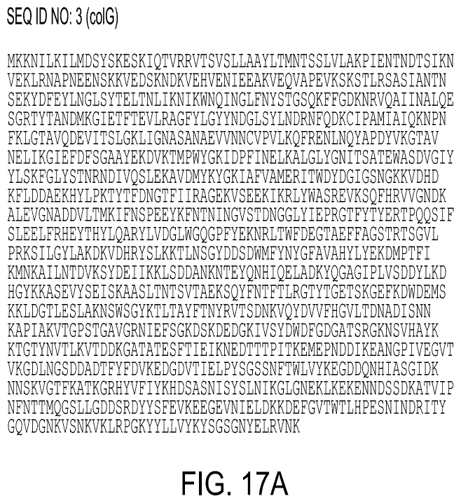

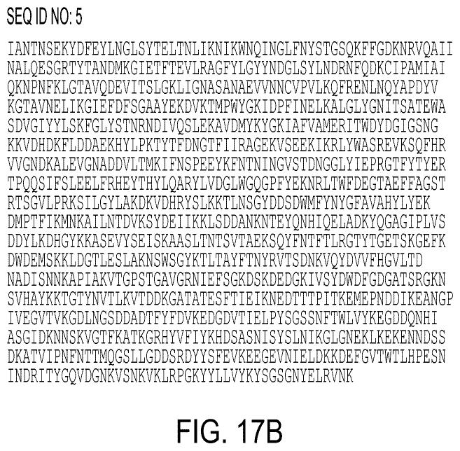

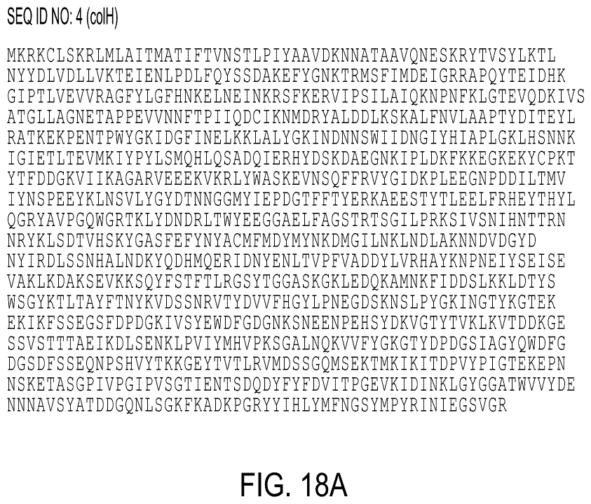

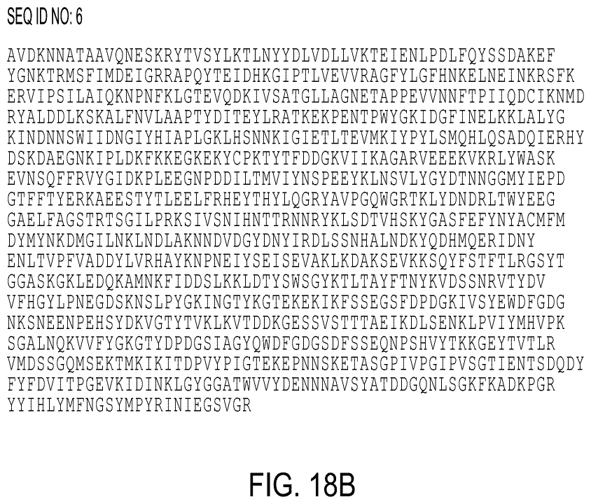

A major source of collagenase is from the fermentation of C. histolyticum. An injectable formulation comprising C. histolyticum collagenase I and collagenase II is sold under the trade name XIAFLEX® and is approved by the U.S. Food and Drug Administration for the treatment of Dupuytren's contracture. Amino acid sequences for collagenase I and collagenase II encoded by the colG and colH genes, respectively, have been described in the literature. For example, colG is described in GenBank Acc. No. D87215 and Matsushita et al. (1999), Journal of Bacteriology 181 (3): 923-933, and colH has been described in GenBank Acc. No. D29981 and Yoshihara et al. (1994), Journal of Bacteriology 176 (21): 6489-6496, the contents of each of which are expressly incorporated by reference herein. The present invention is based partially on sequencing analysis of the genes encoding collagenase I and collagenase II in a C. histolyticum strain (Clone 004 described below in the Examples) which produces and secretes functional collagenase I and collagenase II. The nucleotide sequences of the genes encoding collagenase I and collagenase II were found to be different from the literature-described sequences for C. histolyticum (e.g., GenBank Acc. Nos. D87125 and D29981) (SEQ ID NO: 19 and 20) (FIGS. 9A, 9B, 10A, and 10B).

Collagenase I and collagenase II are metalloproteases and require tightly bound zinc and loosely bound calcium for their activity (Eddie L. Angleton and H. E. Van Wart, Biochemistry 1988, 27, 7406-7412). Collagenase I and collagenase II have broad specificity toward all types of collagen (Steinbrink, D; Bond, M and Van Wart, H; (1985), JBC, 260 p 2771-2776). Collagenase I and collagenase II digest collagen by hydrolyzing the triple-helical region of collagen under physiological conditions (Steinbrink, D; Bond, M and Van Wart, H; (1985), JBC, 260 p 2771-2776). Even though each collagenase shows different specificity (e.g., each has a different preferred amino sequence for cleavage), together, they have synergistic activity toward collagen (Mandl, I., (1964), Biochemistry, 3: p. 1737-1741; Vos-Scheperkeuter, GH, (1997), Cell Transplantation, 6: p. 403-412).

The invention encompasses a recombinant nucleic acid molecule comprising or consisting of a polynucleotide of SEQ ID NO: 1 or the complement of SEQ ID NO: 1. In certain aspects, the recombinant nucleic acid further comprises a heterologous regulatory sequence operably linked to the polynucleotide. The invention further encompasses a recombinant nucleic acid molecule comprising or consisting of a polynucleotide of SEQ ID NO: 2 or the complement of SEQ ID NO: 2. In certain aspects, the recombinant nucleic acid further comprises a heterologous promoter operatively linked to the polynucleotide.

The invention also encompasses recombinant polypeptides encoded by the recombinant nucleic acids described herein. In some aspects, the recombinant polypeptides are encoded by the recombinant nucleic acids comprising or consisting of a nucleotide sequence selected from the group consisting of SEQ ID NO: 1 and SEQ ID NO: 2. In some embodiments, the recombinant polypeptide comprises the amino acid sequence of SEQ ID NO: 3 or SEQ ID NO: 4. In additional embodiments, the recombinant polypeptide comprises the amino acid sequence of SEQ ID NO:5 (the mature collagenase I protein, beginning at Ile 119 of SEQ ID NO: 3 in FIG. 9A and 9B) or SEQ ID NO:6 (the mature collagenase II protein, beginning at Ala 31 of SEQ ID NO: 4 in FIG. 10A and 10B). In yet another embodiment, the recombinant polypeptide consists of the amino acid sequence of SEQ ID NO: 3, SEQ ID NO: 4, SEQ ID NO: 5 or SEQ ID NO: 6.

In yet another embodiment, the invention is directed to a recombinant nucleic acid that encodes a polypeptide which comprises or consists of the amino acid sequence of SEQ ID NO: 3 or SEQ ID NO:4. In a further embodiment, the invention is directed to a recombinant nucleic acid that encodes a polypeptide which comprises or consists of the amino acid sequence of SEQ ID NO: 5 or SEQ ID NO: 6. In a further aspect, the recombinant nucleic acid comprises a nucleotide sequence that encodes a polypeptide of amino acid sequence SEQ ID NO: 3, SEQ ID NO: 4, SEQ ID NO: 5 or SEQ ID NO: 6.

A recombinant nucleic acid is a nucleic acid molecule that contains, in addition to a polynucleotide sequence described herein (for example, the polynucleotide sequence of SEQ ID NO: 1 or SEQ ID NO: 2), a further heterologous coding or non-coding nucleotide sequence. The term “heterologous” means that the polynucleotide originates from a different species or from the same species, however, from another location in the genome than said added nucleotide sequence. Recombinant polypeptides or proteins refer to polypeptides or proteins produced using recombinant techniques, for example, those proteins or polypeptides produced from cells transformed by an exogenous nucleic acid construct encoding the desired polypeptide or protein.

The invention also relates to nucleic acids comprising the polynucleotide sequence of SEQ ID NO: 1 or SEQ ID NO: 2, wherein said nucleic acid is operatively linked to a regulatory sequence. The invention further relates to nucleic acids comprising a polynucleotide that encodes a polypeptide comprising the amino acid sequence of SEQ ID NO: 3, SEQ ID NO: 4, SEQ ID NO: 5 or SEQ ID NO: 6, wherein said nucleic acid is operatively linked to a regulatory sequence. Regulatory sequences include those regulatory sequences which direct constitutive expression of a nucleotide sequence in many types of host cells and/or those which direct expression of the nucleotide sequence only in certain host cells (e. g., tissue-specific regulatory sequences). Non-limiting examples of regulatory sequences are promoters and enhancers. Regulatory sequences also include other expression control elements, for example, those described in Goeddei, Gene Expression Technology: Methods in Enzymology 185, Academic Press, San Diego, CA (1990), the contents of which are expressly incorporated by reference herein. A nucleic acid is “operably linked” to a regulatory sequence when the nucleic acid molecule is linked to the regulatory sequence in a manner which allows expression of the nucleic acid sequence.

A nucleic acid molecule described herein can additionally be fused to a marker sequence, for example, a sequence that encodes a polypeptide to assist in isolation or purification of the polypeptide. Such sequences include, but are not limited to, those which encode a glutathione-S-transferase (GST) fusion protein, those which encode a hemaglutin A (HA) polypeptide marker from influenza, and those which encode hexa-histidine peptide, such as the tag provided in a pQE vector (Qiagen, Inc.). In certain aspects, the invention is directed to a polypeptide comprising an amino acid sequence of SEQ ID NO: 3, SEQ ID NO: 4, SEQ ID NO: 5, or SEQ ID NO: 6, wherein said polypeptide is fused a marker amino acid sequence.

In a further aspect, the invention is directed to a nucleic acid that is a variant of the nucleotide sequence of SEQ ID NO: 1 or SEQ ID NO: 2. A variant nucleic acid is a nucleic acid that includes an nucleotide substitution, addition or deletion relative to nucleotide sequence of SEQ ID NO: 1 or SEQ ID NO: 2. In some aspects, the variant is a nucleic acid that encodes identical or substantially identical amino acid sequences as that of the nucleotide sequences of SEQ ID NO: 1 or SEQ ID NO: 2. As will be understood by the skilled artisan, because of the degeneracy of the genetic code, several different nucleic acid sequences can encode a given protein. For instance, the codons GCA, GCC, GCG and GCU each encode the amino acid alanine. Thus, for example, at every position where a specific amino acid is specified by one codon, the codon can be changed to any of the corresponding codons that encode the same amino acid without altering the amino acid sequence of the encoded polypeptide. One of ordinary skill in the art will understand that each codon in a nucleotide sequence (except AUG, which is the only codon for methionine, and TGG, which is usually the only codon for tryptophan) can be modified to yield a functionally identical molecule.

In certain embodiments, the invention is directed to polypeptide comprising or consisting of amino acid sequence of SEQ ID NO: 3, SEQ ID NO: 4, SEQ ID NO: 5 or SEQ ID NO: 6, wherein one or more amino acids have been deleted or added, wherein the polypeptide possesses the activity of degrading or lysing collagen. In yet an additional embodiment, the polypeptide comprises or consists of the amino acid sequence of SEQ ID NO: 3, SEQ ID NO: 4, SEQ ID NO: 5 or SEQ ID NO: 6, wherein one or more amino acid residues have been replaced with a different amino acid residue, wherein the polypeptide possesses the activity of degrading or lysing collagen and wherein the polypeptide comprises or consists of an amino acid sequence that is different from the amino acid sequences of GenBank Acc. Nos. D87125 (SEQ ID NO: 19) and D29981 (SEQ ID NO: 20). In certain aspects, when an amino acid is replaced, the replacement is a conservative amino acid change. A conservative amino acid change is, for example, substitution of a nonpolar amino acid for another nonpolar amino acid or substitution of a polar amino acid for another polar amino acid or substitution of a positively charged amino acid for another positively charged amino acid, and the like. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

An isolated nucleic acid and an isolated polypeptide are not in the form or environment in which they exist in nature. For example, an isolated nucleic acid is one that is separated from the nucleotides which normally flanks the nucleic acid molecule in nature. Recombinant nucleic acids and recombinant nucleic acids within a vector are also an example of an isolated nucleic acid. Also, isolated nucleic acid molecules include recombinant nucleic acid molecules in heterologous host cells, as well as partially or substantially purified nucleic acid molecules in solution.

As described in more detail below, the invention also encompasses recombinant host cells, such as bacterial cells, fungal cells, plants cells, insect cells, avian cells, amphibian cells and mammalian cells, comprising the nucleic acid molecules described herein.

An expression cassette is a nucleotide sequence which is capable of affecting expression of a structural gene (i.e., a protein coding sequence, such as a collagenase of the invention) in a host compatible with such sequences. Expression cassettes can include a promoter operably linked with the polypeptide coding sequence; and, optionally, with other sequences, e.g., transcription termination signals. Additional factors necessary or helpful in effecting expression may also be used, e.g., enhancers.

The invention also relates to vectors comprising a nucleic acid of the invention. In one embodiment, the nucleic acid is SEQ ID NO: 1 or SEQ ID NO: 2, or a complement thereof. In another embodiment, the nucleic acid is a nucleic acid that encodes a polypeptide having the amino acid sequence of SEQ ID NO: 3, SEQ ID NO: 4, SEQ ID NO: 5 or SEQ ID NO: 6. A “vector” is a nucleic acid molecule capable of transporting another nucleic acid to which it has been linked. A non-limiting example of a vector is a plasmid which is a circular double stranded DNA into which an additional DNA segment can be ligated. Another example of a vector is a viral vector, wherein an additional DNA segment is ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Expression vectors are capable of directing the expression of genes to which they are operably linked. Such expression vectors include, for example, plasmids. The invention encompasses other expression vectors, such as viral vectors (e.g., replication defective retroviruses, adenoviruses and adeno-associated viruses) that are capable of directing gene expression. As will be appreciated by the skilled artisan, the design of the expression vector depends on several factors, such as the choice of the host cell to be transformed, the level of expression of protein desired, and the like. Expression vectors include one or more regulatory sequences which are selected based on the host cell to be used for expression. As discussed above, the regulatory sequence is operably linked to the nucleic acid to be expressed, for example, a nucleic acid of the invention. In some embodiments, the regulatory sequence is a regulatory sequence native to the transformed host cell. An expression vector can comprise one or more selectable markers, including, but not limited to, the gene that encodes dihydrofolate reductase and the genes that confer resistance to neomycin, tetracycline, ampicillin, chloramphenicol, kanamycin and streptomycin resistance.

Prokaryotic and eukaryotic host cells can be transfected by the vectors described herein. Host cells which can be transfected with the vectors of the present invention include, but are not limited to, bacterial cells such as E. coli (e.g., E. coli K12 strains), Streptomyces, Pseudomonas, Serratia marcescens and Salmonella typhimurium, insect cells (baculovirus), including Drosophila, fungal cells, such as yeast cells, plant cells and mammalian cells, such as thymocytes, Chinese hamster ovary cells (CHO), COS cells, and lactococcus lactis cells. In some embodiments, the host cell is a bacterial cell. In yet another embodiment, the host cell is an E. coli strain. In yet an additional embodiment, the host cell is lactococcus lactis cell. Methods for the production of recombinant polypeptides in lactococcus lactis bacteria have been described, for example, in U.S. Pat. No. 7,358,067, the contents of which are expressly incorporated by reference herein. In one embodiment, the host cell is lactococcus lactis and the nucleic acid comprises the nucleotide sequence of SEQ ID NO: 1 or SEQ ID NO: 2 operably linked to pH regulatable promoter P170 and derivatives thereof. The P170 promoter and derivatives thereof have been described in detail in WO 94/16086 and WO 98/10079, the contents of which are incorporated by reference herein.

Ligating the nucleic acid molecule into a gene construct, such as an expression vector, and transforming or transfecting into hosts, either eukaryotic (yeast, avian, insect, plant or mammalian) or prokaryotic (bacterial cells), are standard procedures. A vector described herein can be introduced into prokaryotic or eukaryotic cells using conventional transformation or transfection techniques, including, but not limited to, calcium phosphate or calcium chloride co-precipitation, DEAE-dextran-mediated transfection, lipofection, or electroporation. The polypeptides of the present invention can be isolated or purified (e.g., to homogeneity) from recombinant cell culture by a variety of processes.

The invention encompasses methods of producing a functional collagenase I or collagenase II or a combination thereof comprising culturing a host cell transformed or transfected with a vector comprising a nucleic acid of the invention. The method additionally comprises isolating the polypeptide from the medium or the host cell. A functional collagenase is a polypeptide that has a biological activity of a naturally-occurring collagenase, for example, a collagenase that possesses the ability to degrade collagen.

The polypeptide can be isolated by methods including, but not limited to, anion or cation exchange chromatography, ethanol precipitation, affinity chromatography and high performance liquid chromatography (HPLC), or a combination of any of thereof. The particular method used will depend upon the properties of the polypeptide and the selection of the host cell; appropriate methods will be readily apparent to those skilled in the art.

In some embodiments, the invention is a method of producing collagenase I or collagenase II, said method comprising the steps of (i) constructing a recombinant bacterium comprising the nucleotide sequence of SEQ ID NO: 1 or SEQ ID NO:2, or the polynucleotide encoding the polypeptide sequence of SEQ ID NO: 3, 4, 5 or 6 operably linked to an appropriate regulatory sequence; (ii) cultivating said recombinant bacterium under suitable conditions to express the gene, and (iii) harvesting from the recombinant bacterium, the collagenase I or collagenase II. The collagenase I and collagenase II can be purified by a variety of methods known to those skilled in the art, including dye ligand affinity chromatography, heparin affinity chromatography, ammonium sulfate precipitation, hydroxylapatite chromatography, size exclusion chromatography, ion exchange chromatography, and metal chelation chromatography. In some embodiments, the collagenase I and collagenase II are purified via filtration and column chromatography and the purified collagenase I and II are combined in a ratio of about 1 to 1 using methods described in U.S. Pat. No. 7,811,250, the contents of which are expressly incorporated by reference herein.

Examples of collagen mediated-diseases that can be treated by the compositions (comprising collagenase I, collagenase II, or a combination thereof encoded by the nucleic acids described herein and/or comprising the amino acid sequences of SEQ ID NO: 3 and/or SEQ ID NO: 4) and methods of the invention include, but are not limited to, Dupuytren's disease, Peyronie's disease, frozen shoulder (adhesive capsulitis), keloids, hypertrophic scars, depressed scars, such as those resulting from inflammatory acne; post-surgical adhesions, acne vulgaris, lipomas, and disfiguring conditions such as wrinkling, cellulite formation and neoplastic fibrosis. U.S. Pat. Nos. 6,086,872 and 5,589,171, incorporated herein by reference, disclose the use of collagenase preparations in the treatment of Dupuytren's disease. U.S. Pat. No. 6,022,539, incorporated herein by reference, discloses the use of collagenase preparations in the treatment of Peyronie's disease.

In addition to its use in treating collagen-mediated diseases, a composition comprising a recombinant polypeptide described herein is also useful for the dissociation of tissue into individual cells and cell clusters as is useful in a wide variety of laboratory, diagnostic and therapeutic applications. These applications involve the isolation of many types of cells for various uses, including microvascular endothelial cells for small diameter synthetic vascular graft seeding, hepatocytes for gene therapy, drug toxicology screening and extracorporeal liver assist devices, chondrocytes for cartilage regeneration, and islets of Langerhans for the treatment of insulin-dependent diabetes mellitus. Enzyme treatment works to fragment extracellular matrix proteins and proteins which maintain cell-to-cell contact. Since collagen is the principle protein component of tissue ultrastructure, the enzyme collagenase has been frequently used to accomplish the desired tissue disintegration. In general, the composition of the present invention is useful for any application where the removal of cells or the modification of an extracellular matrix, are desired.

The invention encompasses pharmaceutical compositions comprising a pharmaceutically acceptable carrier and collagenase I and/or collagenase II produced according to a method described herein. In yet another embodiment, the pharmaceutical compositions comprises collagenase I comprising or consisting of the amino acid sequence of SEQ ID NO: 3 or SEQ ID NO: 5. In a further embodiment, the pharmaceutical composition comprises collagenase II comprising or consisting of the amino acid sequence of SEQ ID NO: 4 or SEQ ID NO: 6. In yet another aspect, the pharmaceutical composition comprises a pharmaceutically acceptable carrier and a collagenase I and collagenase II as described herein. In a further aspect, the pharmaceutical composition comprises a pharmaceutically acceptable carrier and the collagenase I and collagenase II at 1:1 mass ratio. The pharmaceutical composition of the present invention comprises an effective amount of a collagenase the present invention formulated together with one or more pharmaceutically acceptable carriers or excipients.

### B. Methods of Detecting the Presence of a Hemolytic Toxin

In some embodiments, the invention encompasses methods of detecting the presence of a hemolytic toxin in a bacterial fermentation, wherein the bacterial fermentation produces a collagenase. In certain aspects, the invention provides a method for releasing a collagenase drug product prior to the therapeutic administration of said collagenase drug substance to a patient comprising detecting the presence of a hemolytic toxin in the drug product production strain. The term “drug product production strain,” “production strain,” “collagenase production strain,” and “bacterial production strain” are used interchangeably and refer to a bacterial strain from which a collagenase is obtained. In other aspects, the invention provides a method for releasing a collagenase drug product prior to the therapeutic administration of said collagenase drug product to a patient, comprising detecting the presence of a hemolytic toxin in the drug product.

As used herein, the phrase “releasing a collagenase drug product” means to confirm the absence of a hemolytic toxin in the collagenase drug product. It is understood that the terms “drug substance”, “drug product” or “collagenase composition” can be used interchangeably. Also as used herein, the terms “hemolysin” and “hemolytic toxin” are used interchangeably, and refer to a toxin that is responsible for the lysis of a red blood cell.

It has been discovered that the collagenase production strain and drug product can be assayed for the presence or absence of hemolytic activity, ensuring that the collagenase drug substance provides a highly reproducible and optimal enzymatic activity and superior therapeutic effect, while lowering the potential for side effects. In accordance with the invention, methods are provided for assaying the production strain or drug product for the secretion or presence of a functional hemolytic toxin that may be co-present with collagenase in the drug product. The invention encompasses a method of assaying a test sample for the presence of a hemolytic toxin, wherein the test sample comprises a bacterial production strain or a collagenase composition, comprising incubating the test sample with red blood cells, followed by detection of lysis of red blood cells.

Specific methods for detecting lysis of red blood cells are described throughout the literature, including, for example, 1) Ryan K J and Ray C G. Principles of laboratory diagnosis. In Sherris medical microbiology: an introduction to infectious diseases. Ryan K J, Ray C G, and Sherris J C (eds.) McGraw-Hill Professional, 2004; 229-260; and 2) Eschbach E et al. Improved erythrocyte lysis assay in microtitre plates for sensitive detection and efficient measurement of hemolytic compounds from ichthyotoxic algae. Journal of Applied Toxicology 21, 513-519 (2001), the contents of each of which are expressly incorporated by reference herein.

In one embodiment of the invention, the method comprises incubating samples of a collagenase production strain, a partially purified collagenase isolated from a collagenase production strain, or a collagenase drug product on a blood agar substrate, and observing the blood agar for zones of clearance after the period of incubation, wherein a zone of clearance indicates the lysis of red blood cells. If the bacterial product strain was tested, the lysis of red blood cells indicates the secretion of a functional hemolytic toxin from the bacterial production strain. If a partially purified collagenase or a collagenase drug product was tested, the lysis of red blood cells indicates the presence of a functional hemolytic toxin in the partially purified collagenase or in collagenase drug product. In certain embodiments, the production strain is a strain of C. histolyticum. The absence of a zone of clearance indicates the absence of a hemolytic toxin. The observed absence of zones of clearance indicate or confirm the absence of hemolytic toxins in the collagenase production strain, in the partially purified collagenase, or in the collagenase drug product, and allow the drug product to be released for therapeutic administration.

In another embodiment, the method comprises incubating red blood cells with extracts taken from a collagenase production strain, or with a partially purified collagenase isolated from a collagenase production strain, or with a collagenase drug product, followed by photometrically analyzing the incubation mixture for the lysis of red cells as indicated by the appearance of hemoglobin in the incubation mixture. A hemolytic toxin will lyse the red blood cells, releasing hemoglobin into the incubated sample. The photometric detection of hemoglobin can provide a sensitive assay for the presence of hemolytic toxins. In one aspect, red blood cells are incubated with extracts taken from a collagenase production strain, or with a partially purified collagenase isolated from a collagenase production strain, or with a collagenase drug product, and then photometrically analyzing the extracts for the presence of hemoglobin at a wavelength of 540 nm. In another aspect, the photometric analysis is performed at a wavelength of 414 nm. In yet another aspect, incubation and photometric analysis can be performed using microtiter plates. The absence of hemoglobin, and thus the absence of hemolytic toxins, would allow the release of the drug product for therapeutic administration to a patient.

Hemolytic toxins as found in C. histolyticum belong to two different families of hemolysins: aerolysin-like hemolysins, and oxygen-labile hemolysins. The aerolysin-like hemolysins are synthesized by the bacterium as inactive preproteins that are secreted into the extracellular environment as inactive protoxins. The inactive protoxins will bind to receptors on a target cell membrane, for example, receptors on a red blood cell where the protoxins are cleaved into their active structures by proteases. Once activated, the toxins oligomerize on the cell surface into a prepore complex, followed by insertion of a beta-barrel into the target cell membrane. The beta-barrel forms a pore in the membrane, allowing the rapid influx of calcium ions into the cell, with toxic consequences to the cell. The alpha toxin of C. histolyticum is most likely an aerolysin-like hemolysin, as it has been discovered to share significant homology with Clostridium septicum alpha toxin, which is a member of the aerolysin-like family of toxins, and which possess hemolytic activity (see, for example, Example 1 below).

Epsilon toxin of C. histolyticum, and tetanolysin of Clostridium tetani (C. tetani), have been described as an oxygen-labile hemolysins [Hatheway C L. Clin Microbiol Rev 3(1): 66-98 (1990)]. Epsilon toxin of C. histolyticum has been discovered to share homology with tetanloysin, which is a member of thiol-activated, beta-barrel, pore-forming toxins with affinity for cholesterol. Such proteins are part of a family of Cholesterol Dependent Cytolysins (CDC). These proteins are secreted by the bacterium into the extracellular environment as water-soluble monomeric proteins where they bind to target cell membranes, mediated by cholesterol binding. The toxin then oligomerizes on the membrane surface to form arcs and ring-like structures that are responsible for cytolysis. The epsilon toxin of C. histolyticum is known to be an oxygen-labile hemolysin, and is similar serologically to those oxygen-labile hemolysins produced by other strains of Clostridium, such as C. tetani, C. novyi, and C. septicum.

In certain aspects, the invention is directed to a method of detecting the presence of C. histolyticum alpha toxin in a bacterial production strain using an assay described herein. In other aspects, the invention is directed to a method of detecting the presence of C. histolyticum alpha toxin in a drug product. In a further aspect, the invention is directed to a method of detecting the presence of C. histolyticum epsilon toxin in a bacterial production strain. In yet another aspect, the invention is directed to a method of detecting the presence of C. histolyticum epsilon toxin in a drug product. In a still further aspect, the invention is directed to a method of detecting the presence of C. histolyticum alpha toxin and epsilon toxin in a bacterial production strain. In an additional embodiment, the invention is directed to a method of detecting the presence of C. histolyticum alpha toxin and epsilon toxin in a drug product.

The invention also encompasses a method of producing a drug product consisting of collagenase I and collagenase II, wherein the collagenase I and II are obtained from C. histolyticum, and wherein the method comprises the steps of fermenting a strain of C. histolyticum in which the absence of a functional, secreted hemolytic toxin has been confirmed by incubating the production strain with red blood cells under conditions suitable for lysis of red blood cells by a hemolytic toxin, wherein lysis of red blood cells indicates secretion of a hemolytic toxin and wherein the absence of lysis of the red blood cells indicates the absence of a hemolytic toxin. In another aspect, the invention is directed to a method of producing a drug product consisting of collagenase I and collagenase II, wherein the collagenase I and II are obtained from C. histolyticum, and wherein the method comprises the steps of confirming the absence of a functional, secreted hemolytic toxin in the drug product by incubating the drug product with red blood cells under conditions suitable for lysis of red blood cells by a hemolytic toxin, wherein lysis of red blood cells indicates secretion of a hemolytic toxin and wherein the absence of lysis of the red blood cells indicates the absence of a hemolytic toxin.

Further aspects of the invention include methods of purifying a crude collagenase composition comprising purifying the composition by filtration and column chromatography followed by confirming the absence of a hemolytic toxin by incubating a sample of the purified composition with red blood cells under conditions suitable for lysis of red blood cells by a hemolytic toxin, wherein lysis of red blood cells indicates secretion of a hemolytic toxin and wherein the absence of lysis of the red blood cells indicates the absence of a hemolytic toxin.

As discussed above, several diseases and conditions are associated with excess collagen deposition and the erratic accumulation of fibrous tissue rich in collagen and can be treated with collagen drug products. Such diseases and conditions are collectively referred to herein as “collagen-mediated diseases”. The invention also encompasses a method of treating a collagen-mediated disease in a patient in need thereof, wherein the composition comprising collagenase is administered to said patient and wherein, prior to said administration, said composition or bacterial production strain is assayed for the presence or absence of hemolytic toxins using a method described herein. Examples of collagen mediated-conditions that may be treated by the compositions and methods described herein include but are not limited to: Dupuytren's disease; Peyronie's disease; frozen shoulder (adhesive capsulitis), keloids; hypertrophic scars; depressed scars such as those resulting from inflammatory acne; post-surgical adhesions; acne vulgaris; lipomas, and disfiguring conditions such as wrinkling, cellulite formation and neoplastic fibrosis. In certain aspects, the assayed composition is administered to a patient to treat Peyronie's or Duputyren's diseases or adhesive capsulitis.

With respect to the production strain that can be assayed according to a method of the invention, it is known, for example, that collagenase is expressed by bacteria that are members of the genera Actinobacillus, Actinomadura, Bacillus, Bacteriodes, Bifidobacterium, Brucella, Capnocytophaga, Clostridium, Enterococcus, Escherichia, Eubacterium, Flavobacterium, Fusobacterium, Peptococcus, Peptostreptococcus, Porphyromonas, Prevotella, Proteus, Pseudomonas, Serratia, Staphylococcus, Streptomyces, Streptococcus, Treponema, and Vibrio. In one embodiment of the invention, the production strain is selected from the above listed genera. In another embodiment, the production strain is an E. coli strain, including forms of E. coli that have been transformed with recombinant forms of collagenase I and collagenase II. In a more preferred embodiment, the production strain is a C. perfringens strain. In a most preferred embodiment, the production strain is a C. histolyticum (C. his) strain.

In certain aspects, the production strain produces a collagenase composition comprising a mixture of collagenase I and collagenase II. In a further embodiment, the production strain used to produce a mixture of collagenase I and collagenase I is C. histolyticum. In another embodiment, the collagenase drug product comprises a mixture of highly purified C. histolyticum collagenase I and collagenase II in a mass ratio of about 1 to 1.

Kits for testing for the presence of hemolysins in a sample are also presented, wherein a hemolysin is a substance that causes lysis of red blood cells. The kits allow the identification of test substances that are hemolytic, or contain, hemolysins. Test substances include, but are not limited to, chemical, biological, and radiation-emitting substances. In one embodiment, the kit comprises materials for testing for the presence of hemolysins in a test sample including, for example, a kit comprising red blood cells and related test materials. In another embodiment, the kit comprises a petri dish comprised of blood agar, a positive control, and a negative control comprised of a bacterial strain wherein the hemolytic genes are mutated or knocked out, and wherein no functional hemolytic proteins are produced. In yet another embodiment, the kit comprises red blood cells, microtiter plates, a positive control, and a negative control comprised of the drug product.

As will be understood, the inventive kits and methods can be used to detect the presence or absence of hemolysins in collagenase compositions, wherein the collagenase is obtained from a bacteria.

The crude collagenase obtained from C. histolyticum can be purified by a variety of methods known to those skilled in the art, including dye ligand affinity chromatography, heparin affinity chromatography, ammonium sulfate precipitation, hydroxylapatite chromatography, size exclusion chromatography, ion exchange chromatography, and metal chelation chromatography. Crude and partially purified collagenase is commercially available from many sources including Advance Biofactures Corp., Lynbrook, New York. Methods of purification of crude collagenase obtained from C. histolyticum are also described in U.S. Pat. No. 7,811,560, the contents of which are expressly incorporated herein by reference. In certain embodiments, the purification procedure comprises the steps of: a) filtering the crude harvest through a MUSTANG Q anion-exchange capsule filter; b) adding ammonium sulphate; preferably to a final concentration of 1M; c) filtering the crude harvest; preferably through a 0.45 μm filter; d) subjecting the filtrate through a HIC column; preferably a phenyl sepharose 6FF (low sub); e) adding leupeptin to the filtrate; preferably to a final concentration of 0.2 μm to post HIC eluted product; f) removing the ammonium sulfate and maintaining leupeptin for correct binding of collagenase I and collagenase II with buffer exchange by TFF; preferably with buffer exchange by TFF; g) filtering the mixture of step; (f) preferably through a 0.45 μm filter; h) separating collagenase I and collagenase II using Q-Sepharose HP; i) preparing TFF concentration and formulation for collagenase I and collagenase II separately; wherein TFF is a tangential flow filtration using 10 and/or 30 K MWCO (molecular weight cut-off) PES or RC-polyethersulfone or regenerated cellulose filter membranes (TFF provides a means to retain and concentrate select protein and exchange the protein from one buffer solution into another); and j) filtering through a 0.2 μm filtration system.

C. C. histolyticum Alpha, Beta, Delta, Epsilon and Gamma Toxins

The amino acid sequences of the alpha, delta and epsilon toxins of C. histolyticum Clone 004 are shown in the Figures and are SEQ ID NO: 8, SEQ ID NO: 12 and SEQ ID NO: 16, respectively. The nucleotide sequences of the alpha, delta and epsilon toxins of C. histolyticum Clone 004 are also shown in the Figures and are SEQ ID NO: 21, SEQ ID NO: 22, and SEQ ID NO: 23, respectively. Each of the amino acid sequences of SEQ ID NO: 8, SEQ ID NO: 12, SEQ ID NO: 16 have sequence characteristics that render these proteins non-functional and/or unsecreted.

For the gamma toxin (clostripain), there are only three amino acid differences when compared to the model protein (see Examples section) and none of the amino acid residues which are found to differ in the C. histolyticum Clone 004 gamma toxin have been identified as essential for activity. Thus, it is predicted that the C. histolyticum Clone 004 gamma toxin (having the amino acid sequence of SEQ ID NO: 18) is secreted and functional. The nucleotide sequence of the C. histolyticum Clone 004 gamma toxin is SEQ ID NO: 24.

As discussed above, the beta toxins having amino acid sequences of SEQ ID NO: 3 and SEQ ID NO: 4 are fully functional.

As will be understood, one or more mutations (for example, deletion or addition of one or more amino acid residues or nucleic acid residues) can be introduced into the nucleotide and/or amino acid sequences of C. histolyticum alpha, beta, epsilon or gamma toxins (SEQ ID NO: 8, SEQ ID NO: 12, SEQ ID NO: 16 and SEQ ID NO: 18, SEQ ID NO: 21, SEQ ID NO: 22, SEQ ID NO: 23 and SEQ ID NO: 24). In certain aspects, one or mutations are introduced in order to improve or impair the activity, function, production and/or secretion of the toxin. In one embodiment, a mutation can be introduced that renders the alpha, beta, and/or epsilon toxins functional and/or secreted. In another embodiment, the sequence of the gamma toxin (SEQ ID NO: 18) can be mutated so as to render the protein non-functional and/or unsecreted.

Also encompassed by the present invention are methods of producing antibodies against C. histolyticum or a C. histolyticum toxin comprising administering to a subject an effective amount of a composition comprising a protein or peptide, wherein said protein or peptide comprises an amino acid sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 3, SEQ ID NO: 4, SEQ ID NO: 5 and SEQ ID NO: 6, or a fragment or variant thereof, or a combination of any of thereof. In addition, the present invention includes methods of stimulating an immune response to a C. histolyticum toxin comprising administering to a subject an effective amount of a composition comprising a protein or peptide comprising an amino acid sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 3, SEQ ID NO: 4, SEQ ID NO: 5 and SEQ ID NO: 6, or a fragment or variant thereof, or a combination of any of thereof. The invention also includes a vaccine comprising an effective amount of a protein or peptide comprising an amino acid sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 3, SEQ ID NO: 4, SEQ ID NO: 5 and SEQ ID NO: 6, or a fragment or variant thereof, or a combination of any of thereof. The protein or peptide comprising an amino acid sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 3, SEQ ID NO: 4, SEQ ID NO: 5 and SEQ ID NO: 6, or a fragment or variant thereof can be produced by a C. histolyticum strain or can be a recombinant protein or peptide.

### D. Pharmaceutical Compositions Comprising Collagenase and Methods of Treatment

The invention described herein encompass pharmaceutical compositions comprising the protein sequences and recombinant proteins and also, pharmaceutical compositions comprising a collagenase drug product assayed according to methods described herein. As used herein, the term “pharmaceutically acceptable carrier or excipient” means a non-toxic, inert solid, semi-solid or liquid filler, diluent, encapsulating material or formulation auxiliary of any type. “Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder. A “therapeutically effective amount” or an “effective amount” is an amount which, alone or in combination with one or more other active agents, can control, decrease, inhibit, ameliorate, prevent or otherwise affect one or more symptoms of a disease or condition to be treated. In the context of producing an immune response or in the preparation of a vaccine, an “effective amount” encompasses an amount effective to produce an immune response, including the generation of antibodies against an antigen.

Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, perfuming agents, preservatives and antioxidants can also be present in the composition, according to the judgment of the formulator.

Collagenase compositions can also be prepared by mixing either a specific number of activity units or specific masses of the preferably purified enzymes. Collagenase activity can be measured by the enzyme's ability to hydrolyze either synthetic peptide or collagen substrate. Those skilled in the art will recognize that enzyme assays other than those disclosed herein may also be used to define and prepare functionally equivalent enzyme compositions. Collagenase activity can be described, for example, in SRC units. One SRC unit will solubilize rat tail collagen into ninhydrin reaction material equivalent to 1 nanomole of leucine per minute, at 25° C. and pH 7.4. In certain embodiments of the present invention, collagenase activity is described in ABC units. This potency assay of collagenase is based on the digestion of undenatured collagen (from bovine tendon) at pH 7.2 and 37° C. for 20-24 hours. The number of peptide bonds cleaved is measured by reaction with ninhydrin. Amino groups released by a trypsin digestion control are subtracted. One net ABC unit of collagenase will solubilize ninhydrin reactive material equivalent to 1.09 nanomoles of leucine per minute. 1 SRC unit equals approximately 6.3 ABC units.

In certain aspects, the drug substance for injectable collagenase consists of two microbial collagenases, referred to as Collagenase AUX I and Collagenase ABC I and Collagenase AUX II and Collagenase ABC II. It is understood that the terms “Collagenase I”, “ABC I”, “AUX I”, “collagenase AUX I”, and “collagenase ABC I” mean the same and can be used interchangeably. Similarly, the terms “Collagenase II”, “ABC II”, “AUX II”, “collagenase AUX II”, and “collagenase ABC II” refer to the same enzyme and can also be used interchangeably. These collagenases are secreted by bacterial cells. They are isolated and purified from C. histolyticum culture supernatant by chromatographic methods. Both collagenases are special proteases and share the same EC number (E.C. 3.4.24.3).

Collagenase AUX I has a single polypeptide chain consisting of approximately 1000 amino acids with a molecular weight of 115 kDa. Collagenase AUX II has also a single polypeptide chain consisting of about 1000 amino acids with a molecular weight of 110 kDa.

In some embodiments, the drug substance (collagenase concentrate) has an approximately 1 to 1 mass ratio for collagenase AUX I and AUX II. In one embodiment, the collagenase concentrate has an extinction coefficient of 1.528.

The pharmaceutical compositions of this invention can be administered parenterally, topically, or via an implanted reservoir. The term “parenteral,” as used herein, includes subcutaneous, intracutaneous, intravenous, intramuscular, intraarticular, intraarterial, intrasynovial, intrasternal, intrathecal, intralesional and intracranial injection or infusion techniques. In a preferred embodiment, the composition is injected into the affected tissue. In the case of Peyronie's or Duputyren's diseases or adhesive capsulitis, the composition is injected into the cord of the hand or the Peyronies' plaque. The term “local administration” is defined herein to embrace such direct injection into the affected tissue. In certain aspects, the pharmaceutical composition of the invention is an injectable formulation. In certain additional aspects, the pharmaceutical composition is a topical formulation.

Furthermore, depending on the treatment, improved results can, in some circumstances, be obtained by immobilizing the site of injection after administration of the pharmaceutical composition. For example, the site of administration (e.g., the hand), can be immobilized for 4 or more hours.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids, such as oleic acid, are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The sterile solutions may also be lyophilized for later use.

In some embodiments, the composition comprising collagenase is a lyophilized, injectable composition formulated with sucrose, Tris at a pH level of about 8.0. Generally, a source of calcium is included in the formulation, such as calcium chloride.

Dosage forms for topical or transdermal administration of a pharmaceutical compositions of this invention include ointments, pastes, creams, lotions, gels, powders, solutions, sprays, inhalants or patches. The active component is admixed under sterile conditions with a pharmaceutically acceptable carrier and any needed preservatives or buffers as may be required.

The ointments, pastes, creams and gels may contain, in addition to a polypeptide of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the polypeptides of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of an active agent to the body. Such dosage forms can be made by dissolving or dispensing the active agent in the proper medium. Absorption enhancers can also be used to increase the flux of the polypeptide across the skin. The rate can be controlled by either providing a rate-controlling membrane or by dispersing the polypeptide of the invention in a polymer matrix or gel.

Therapeutic administration of the pharmaceutical may be parenterally, topically, or via an implanted reservoir. The term parenteral as used herein includes subcutaneous, intracutaneous, intravenous, intramuscular, intraarticular, intraarterial, intrasynovial, intrasternal, intrathecal, intralesional and intracranial injection or infusion techniques. The term “local administration” is defined herein to embrace such direct injection. In one embodiment, therapeutic administration of the pharmaceutical composition is by injection.

Therapeutic administration of the pharmaceutical in dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required.

The ointments, pastes, creams and gels may contain, in addition to an active compound of the drug product, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of the drug product, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The invention will be better understood in connection with the following examples, which are intended as an illustration only and not limiting of the scope of the invention. Various changes and modifications to the disclosed embodiments will be apparent to those skilled in the art and such changes and modifications may be made without departing from the spirit of the invention and the scope of the appended claims.

### EXAMPLES

### Example 1: C. histolyticum Genome Sequencing and Toxin Sequence Analysis

A dearth of scientific studies related to the C. histolyticum alpha, delta and epsilon (α, δ, and ε toxins) has resulted in limited knowledge about the protein structure of these toxins. To address this knowledge deficit, a genome sequencing initiative was undertaken to more fully understand the production organism with particular focus on the identification of putative toxin genes. As a consequence of this effort, the complete genome of the Collagenase Clostridium Histolyticum production strain (Clone 004) (Auxilium Product Operation, Malvern, PA) has only recently been generated, representing apparently, the first time that the genome sequence of any C. histolyticum strain has been reported.

There were three fundamental steps involved in the genome sequence project. First, genomic DNA was extracted from a Clone 004 cultivation and forwarded to Creative Genomics for sequencing (Shirley, NY, USA). The genome sequence of C. histolyticum Clone 004 was obtained using industry standard methods. Second, the results obtained from the genome sequence were analyzed using standard bioinformatics methods (BLAST analysis) in order to query the sequence information against genome sequence databases. This second stage resulted in the assignment of protein information for each C. histolyticum gene that was identified. The use of two databases ensured a comprehensive evaluation but also served as a second source to verify the protein assignment. The third step in the project was a comparative analysis of the C. histolyticum putative toxin sequence with the protein assigned automatically by the BLAST analysis.

i. C. histolyticum Genome Sequencing and Identification of Model Proteins

Samples of genomic DNA isolated from an expansion of C. histolyticum (CLH) WCB derived from Clone 004 was forwarded to Creative Genomics (Shirley, NY, USA) for genome sequencing. Creative Genomics employed standard methods used for sequence determination of genomic DNA samples submitted by clients. The genome sequence was generated from Roche/454 GS-FLX system with titanium chemistry (fragment sequencing) accompanied with Illumina/Solexa Genome Analyzer. The ANI 3730x1 was employed to accomplish genome finishing by primer walking. The entire genome sequence of 2,842,906 base pairs with a 29.44% GC content was completed and these values were typical of the genome size and GC content obtained for other Clostridial genomes. Each of the 2,887 open reading frames (ORFs) identified was assigned a unique CLH number. Each of the putative 2,887 genes was further investigated using BLAST analysis of the GenBank and SwissProt databases resulted in the tentative assignment of the loci for beta, gamma, alpha, and epsilon toxins. The results of the initial assessment are presented in Table 1. Thus, the assignment of model proteins was completed as a result of an automated analysis via a comprehensive search of two databases. The model protein assignment was not influenced by operator interpretation.

TABLE 1
Assignments of Model Proteins for Putative CLH
Toxins based upon Comparison with Two Sequence Databases

| Toxin | CLH Name | Common Name | Model Protein |
|---|---|---|---|
| alpha | CLH-2834 & | Lethal factor | Aerolysin/Hemolysin |
| | 2835 | | (C. septicum alpha toxin) |
| beta | CLH_1768 & | Collagenase I | Collagenase I from colG |
| | 1769 | | |
| beta | CLH_2116 | Collagenase II | Collagenase II from colH |
| epsilon | CLH_1920 | Oxygen labile | C. perfringens perfringolysin |
| | | hemolysin | C. tetani tetanolysin |
| gamma | CLH_1861 | Clostripain | C. histolyticum clostripain |

An inspection of the BLAST analysis results of the C. histolyticum genome did not reveal an ORF coding for an elastase. However, proteases have been classified by MEROPS (MEROPS.sanger.ac.uk/) based upon the criterion of the most prominent functional group in the active site of those proteases. Using this MEROPS based functional approach, an elastase falls into the M4 peptidase family of which thermolysin (EC 3.4.24.27) is the best studied member of the family and is the classical model for such proteases. Using this knowledge, a re-inspection of the BLAST analysis output suggested that C. histolyticum possesses a single ORF that shares significant homology with thermolysin. Therefore, the putative delta toxin gene within C. histolyticum has been assigned as a homolog of B. proteolyticus thermolysin.

The results of the initial assessment are presented in Table 2 below based upon comparison with two sequence databases.

TABLE 2
Assignments of Model Proteins for Putative CLH Toxins

| Toxin | CLH Name | Common Name | Model Protein |
|---|---|---|---|
| alpha | CLH 2834 & | Lethal factor | C. septicum alpha toxin |
| | 2835 | | |
| beta | CLH_1768 & | Collagenase I | Collagenase I from colG |
| | 1769 | | |
| beta | CLH_2116 | Collagenase II | Collagenase II from colH |
| delta | CLH_2576 | Elastase | B. thermoproteolyticus |
| | | | thermolysin |
| epsilon | CLH_1920 | Oxygen labile | C. perfringens perfringolysin |
| | | hemolysin | |
| gamma | CLH_1861 | Clostripain | C. histolyticum clostripain |

ii. Protein Sequence Alignments and Analysis

To identify the signal peptide, the sample and control sequences were analyzed in a program termed SignalP identify potential signal peptide sequences (Nielsen (2004), J. Glasgow et al., eds., Proc. Sixth Int. Conf. on Intelligent Systems for Molecular Biology, 122-130. AAAI Press, 1998). A signal peptide is usually located within the first seventy amino acids (or the N-terminus region) of the protein sequence and acts as a signal sequence for the enzyme to be secreted. The signal peptide is cleaved and the resulting protein sequence is the mature protein. Using SignalP, the user can identify the signal peptide cleavage site location in order to identify the N-terminus of the mature protein. For some sample sequences, in particular alpha toxin and beta toxin (AUX-I), however, only the mature protein was identified, not the entire protein sequence including the signal peptide sequence. Further examination revealed that the sequence fragmentation procedure employed separated the signal peptide sequence from the mature protein. The mature protein and signal sequences were reassembled and processed through the alignment tool.

Once all the protein sequences were collected, pair wise sequence alignments were constructed using MATLAB 7.0.10 (The Math Works, Inc., 2010). Pair wise sequence alignments are direct comparisons of two sequences to determine the similarities and differences between two sequences. Both control and sample sequences were uploaded into MATLAB and an alignment was made using the Needleman-Wunsch algorithm and BLOSUM50 scoring matrix. The algorithm and scoring matrix assist in assembling the alignment as the algorithm dictates the value of each amino acid match or mismatch based off of the scoring matrix and incorporates gap values when necessary. Gaps can occur for multiple reasons, including, but not limited to, two sequences having varying lengths and to ensure that the appropriate amino acids are matching up to one another. The scoring matrix is based off of substitution rates observed frequently among sequences and serves to rate the similarity or dissimilarity between two sequences (National Center for Biotechnology Information).

The Hatheway (1990) review (Clin Microbiol Rev 3:66-98) indicated that all five toxins were secreted proteins (exosubstances) and all five toxins had identifiable functionality. This information was used to conduct analysis of the putative CLH toxins. To analyze the protein function of the putative CLH toxins, a number of model proteins were selected based upon literature findings and BLAST results. The controls were downloaded from the National Center for Biotechnology Information (NCBI) in Fasta format.

### 1. Alpha (a) Toxin

Sparse information related to C. histolyticum alpha toxin following the work of Bowen (1952) (Yale J Biol Med 25: 124-138) exists in the literature. Thus, the interrogation of the genome sequence for putative toxin genes was of interest. A preliminary analysis of the genome suggested that C. histolyticum possessed a single ORF that shares significant amino acid homology with C. septicum alpha toxin as determined by BLAST analysis of two databases. Therefore, the putative alpha toxin gene within C. histolyticum has been assigned as a homolog of C. septicum alpha toxin. Studied extensively by the Rodney K. Tweten laboratory, C. septicum alpha toxin was classified as a member of the aerolysin-like family of toxins. Notably, C. septicum alpha toxin does possess haemolytic activity (Ballard et al. (1992), Infect Immun 60: 784-790; Melton-Witt et al. (2006), Biochem 45: 14347-14354) and is distinct from oxygen labile hemolysins as described for C. histolyticum ε toxin (Hatheway (1990), Clin Microbiol Rev 3: 66-98).

The C. septicum alpha toxin is elaborated as an inactive preproprotein which is processed to the extracellular environment as an inactive protoxin. The protoxin then binds to receptors on the cell membrane where they are cleaved into their active structures by proteases (usually furin). A furin consensus site within the toxin is essential for activation by eukaryotic proteases. The activation involves the cleavage of 40-45 amino acids from the C-terminus. Absent the C-terminal cleavage the C. septicum alpha toxin is not functional. Full length C. septicum alpha toxin is haemolytic (Ballard et al. 1992). The active toxin is approximately 41.3 kDa (Gordon et al. 1997). Once activated, the toxins oligomerize on the cell surface into a prepore complex followed by insertion of a beta-barrel into the membrane.

The model C. septicum kalpha toxin consists of three distinct domains termed: D1, D2, and D3. The D1 domain is involved with receptor binding and oligomerization, while the D2 domain contributes to amphipathic-hairpin structure. The D3 domain has a D3 propeptide region that includes a short carboxyl-terminal peptide cleaved at the known AT activation site (R398) and functions as an intramolecular chaperone that prevents premature oligomerization of the alpha toxin. Using saturation mutagenesis, single amino acid substitutions within each domain have allowed the determination of those residues essential for biological activity (Melton-Witt et al., 2006). Importantly, the functional assay utilized a cell viability assay to determine LD₅₀ doses. Thus, the relative effect of single amino acid substitutions within the entire coding region was assessed using a functional assay.

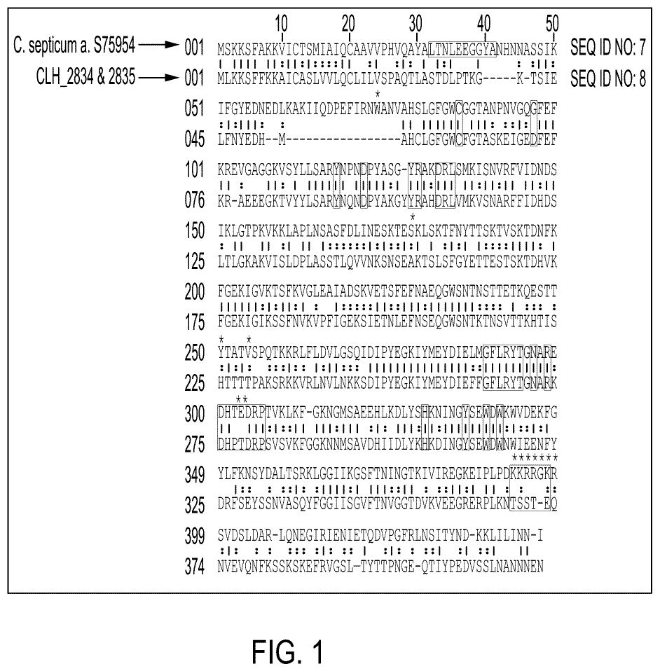

To further understand the primary structure of the CLH alpha toxin, the protein alignment, performed in MATLAB, of the model protein (C. septicum alpha toxin) was made with the CLH alpha toxin. The results are presented in FIG. 1.

The translated CLH putative alpha toxin has an identifiable signal sequence and has a very high probability of being a secreted protein. Thus, the first criterion of an exosubstance is achieved. There is a 75% positive homology between the C. septicum alpha toxin protein sequence and the CLH alpha toxin protein sequence. Multiple regions of high homology were identified between the model alpha toxin and the CLH putative alpha toxin. Such regions and essential amino acid residues are highlighted in green shading in FIG. 1.

Notably, the alignment shows multiple differences in essential amino acid residues that, based on the work of Melton-Witt et al. (2006) (Biochem 45: 14347-14354), individually render the CLH_2834 & 2835 protein non-functional. Beginning with the N-terminal region of the mature protein, a 17 amino acid sequence region is missing in the CLH alpha toxin sequence which is located about 20 amino acids downstream from the putative signal peptide cleavage site. Within this 17 amino acid stretch, a W74 residue on C. septicum alpha toxin has been identified as a critical residue in loop 1 (L1). The lack of 17 amino acids from the D1 domain in the CLH sequence version suggests an altered structure for this domain relative to a wild type and a disruption of the receptor binding functionality.

Within the C-terminal region of the protein, several amino acid residue changes also render the CLH protein non-functional. The amino acid T302 in the C. septicum alpha toxin was replaced by Proline in the CLH alpha toxin. Residue E303 in the C. septicum alpha toxin is replaced by Threonine in the CLH alpha toxin. The studies of Melton-Witt et al. (2006) (Biochem 45: 14347-14354) indicated that each of these modifications will individually result in 0% lethality. Of note is the comparison of the activation site, or furin cleavage site, between the two sequences. The C. septicum alpha toxin exhibits a furin consensus cleavage site beginning with K391 and terminating at R398. This region fits the consensus furin cleavage sequence Arg-X-Lys/Arg-Arg (SEQ ID NO: 31), although the minimal cleavage sequence is Arg-X-X-Arg (SEQ ID NO: 32). The CLH putative alpha toxin has a Glutamine residue instead of Arginine in the analogous R398 position. Thus, the C. septicum activation site possesses the amino acid sequence, DKKRRGKRSVDS (SEQ ID NO: 26), with R398 identified as a critical residue. The CLH alpha toxin homologous sequence in the D3 peptide is NTSST-EQNVEV (SEQ ID NO: 27); beginning with N367 of SEQ ID. NO. 8. Therefore, the putative C. histolyticum alpha toxin furin cleavage site appears to be non-functional, and this protein, even if expressed, could not be processed by contact with eukaryotic cells furin protease to generate a functional toxin. The findings of the comparative amino acid sequence analysis are summarized in Table 3.

TABLE 3
Summary of Amino Acid Sequence
Alignment Comparison for Putative CLH alpha toxin

| Protein | | |
|---|---|---|
| C. septicum α toxin | CLH 2834 & | |
| Essential Amino Acid Residue | 2835 | Effect on Function |
| W 74 | Missing | Receptor binding disrupted |
| T302 | P | Lack of lethality |
| E303 | T | Lack of lethality |
| K391-R398 | T---Q | Incapable of activation |

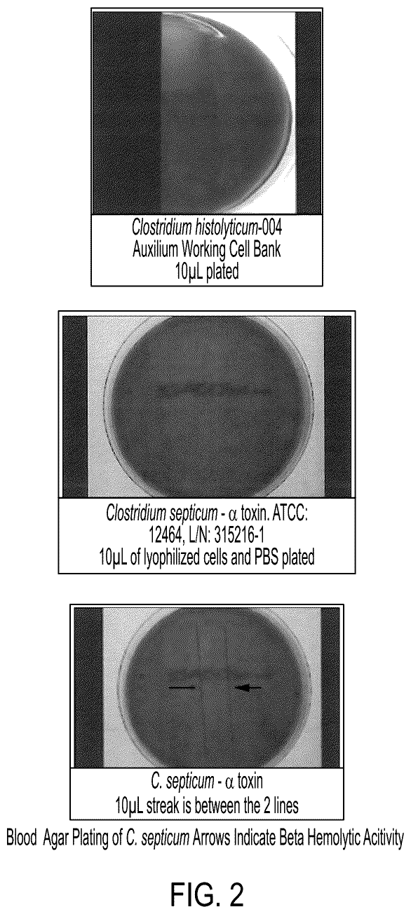

The summary of the sequence alignment analysis suggests that the putative CLH alpha toxin possess a significant number of amino acid residues differences that would make the mature protein non-functional. The phenotypic linkage to functionality for alpha toxin is the demonstration of haemolytic activity. Importantly, the Collagenase Clostridium Histolyticum production strain does not exhibit haemolytic activity when plated on blood agar. The results of a Blood agar hemolytic assessment are illustrated in FIG. 2.

Panel A of FIG. 2 shows the results obtained when a sample of C. histolyticum Clone 004 cell expansion is cultivated on Blood agar. There is no evidence of any beta hemolytic phenotype. In contrast, panel B of FIG. 2 shows the results obtained when a sample of C. septicum is cultivated on Blood agar. There is clear evidence of beta hemolysis that extends well beyond the area of sample application as indicated in Panel C. The images presented do not adequately represent the qualitative difference observed when one views the test articles. The appearance of beta hemolysis is easily discernable and the complete lack of any hemolysis in the C. histolyticum plate stands in stark contrast to the broad zone of hemolysis noted when the C. septicum culture (producer of a toxin) is inspected.

### 2. Delta (8) Toxin

Hatheway et al. (1990) (Clin Microbiol Rev 3:66-98) has defined the δ toxin of C. histolyticum as an elastase, primarily based on the initial research communication by Takahashi, et al. (1970) (BBRC 39: 1058-1064). No further substantial studies on this toxin have apparently been published since then. Four fractions demonstrating elastase activity were isolated from C. histolyticum by Takahashi et al. using differential ultrafiltration. The primary focus was on a fraction which passed through membranes of nominal 50 kDa cut-off membranes but was retained by membranes with a nominal 10 kDa cut-off.

Thermolysin is a zinc metalloprotease with a mature enzyme molecular weight of 34.6 kDa. Importantly, thermolysin is a model protein for a class of proteins that contain a presequence employed in secretion (signal peptide) but also a lengthy prosequence of approximately 200 amino acid residues that is two thirds the size of the mature protein. Thermolysin-like enzymes are elaborated as inactive preproproteins with the prosequence serving a role as an inhibitor of the mature enzyme and also as a chaperone to ensure proper folding of the enzyme (O'Donohue et al. (1996), JBC 271: 26477-26481). The prosequence is autocatalytically removed by the mature enzyme portion of the molecule in the extracellular environment. Thus, the maturation pathway for thermolysin-like enzymes includes: a secretion step, the presence of a pro-mature form in the extracellular matrix, the cleavage of the prosequence, and the presence of a mature, active enzyme.

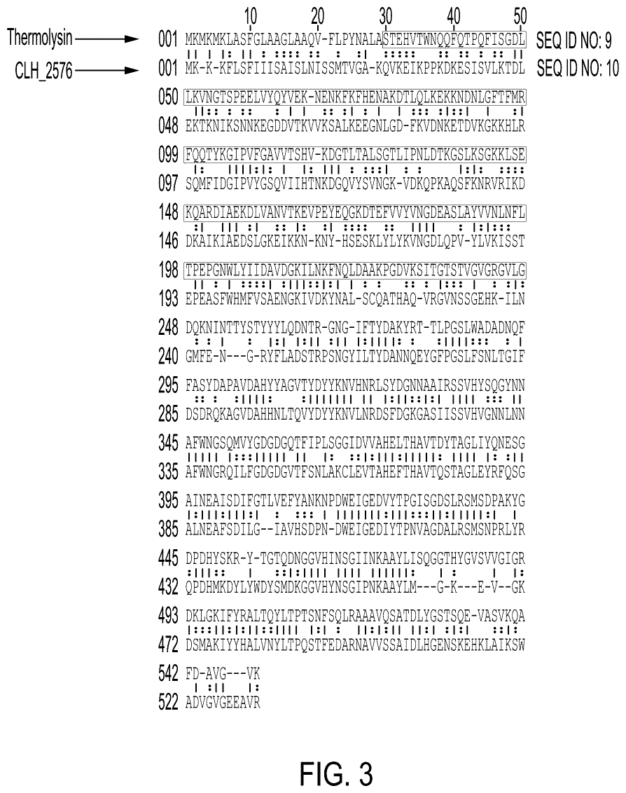

The gene sequence alignment for thermolysin and CLH_2576, the putative C. histolyticum delta toxin, is illustrated in FIG. 3. This image displays the full length prepromature amino acid sequence as a single unit that is theoretically transcribed as a single polypeptide. The initial 28 amino acids at the N-terminus of thermolysin are shown juxtaposed to the green shaded prosequence which terminates at Ser232. The unshaded mature amino acid sequence begins with Ile233. Using the SignalP program, the thermolysin and the CLH_2576 polypeptides are predicted to be secreted. The translated putative C. histolyticum delta toxin has an identifiable signal sequence and a very high probability of being a secreted protein. There is a 65% positive homology between the thermolysin protein sequence and the CLH delta toxin protein sequence.

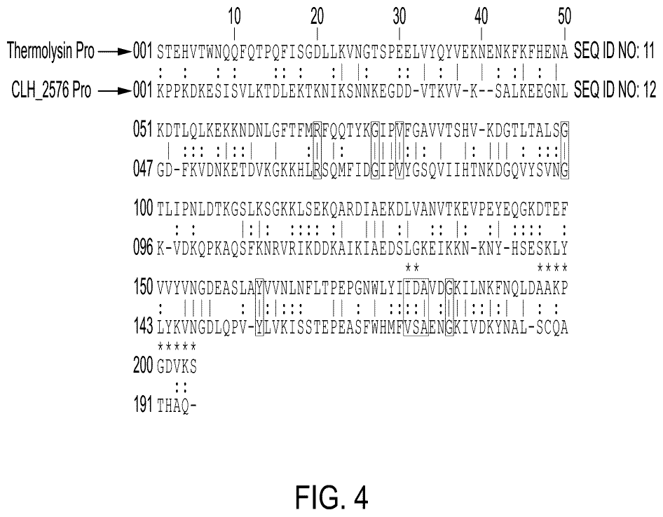

To understand the nature of the pro and mature forms of both proteins, the individual regions were analyzed as distinct sequences with regards to functionality. The prosequence alignment is depicted in FIG. 4. There is a 57% positive homology between the two prosequence forms. A recent review of the primary structural analysis of the prosequences of over 100 thermolysin-like proteases was conducted by Demidyuk et al. (2008) (Protein J 27: 343-354). These investigators noted that considerable variability existed within the prosequences, alternatively termed precursors or propeptides. The prosequences were more tolerant to mutations compared to the corresponding mature enzymes. Nevertheless, regions exhibiting a high degree of conservation and substitutions in key residues were noted which may dramatically alter the function. The residues shaded green in FIG. 3 identify those amino acid residues that are critical for the prosequence to function. No differences are noted between the thermolysin and CLH_2756 sequences. Two residues corresponding to Ile183 and Arg184 in the thermolysin sequence are shaded yellow; however, the substitutions in the CLH_2756 sequence are similar amino acids that likely do not result in any alteration of function.

Importantly, there is a region of non-homology at the C-terminus of the prosequences as illustrated by the yellow shading of the CLH_2756 sequence beginning with Ser185. This region is the site of autocatalysis and suggests that the CLH_2756 sequence is not an acceptable substrate for cleavage by the active site of the mature enzyme. The criticality of the amino acid residues around the cleavage site was investigated by Wetmore et al. (1994) (Mol Microbiol 12: 747-759), using Bacillus cereus thermolysin-like neutral protease as the model enzyme. These investigators determined that the processing was particularly sensitive to the nature of the amino acid three residues upstream from the cleavage site. A consensus sequence was identified for the sequence around the proprotein processing site and alterations in key residues resulted in the non-export or nonprocessing of the protein to a mature, functional enzyme. Key features of the consensus sequence were: the presence of a non-polar residue in position P3 (Gly, Ala, Ile, Leu, or Val), a polar residue or Pro in position P1 (Pro, Ser, His, Glu), and a non-polar residue in position P1′. Additionally, the prothermolysin maturation has been shown to occur between a serine and isoleucine residue (O'Donohue et al. (1994), Biochem J. 300: 599-603). To explore the sequence alignment around the cleavage site, a comparative sequence assessment of the proprotein processing sites for thermolysin and for CLH_2576 can be made by inspection. It is apparent that the CLH_2576 amino acid sequence in the proprotein processing area does not contain the appropriate amino acid arrangement to allow autocatalysis. When one conducts a theoretical exercise to interrogate the CLH_2576 amino sequence to determine if the proprotein processing site is reasonably close to the predicted site based on sequence alignment, it is clear that no adjustment allows the proper amino acid sequence to be identified. Shifting the proprotein processing site 2 residues to the C-terminal side allows for the proper arrangement of amino acids that do not violate the Wetmore et al. rules. However, the Ser-Ile rule of O'Donohue et al. (1994) (Biochem J. 300: 599-603) is not present. Thus, it is concluded that the proprotein form of the CLH_2576 polypeptide is not a suitable substrate for autocatalysis. The net effect is that the mature, active enzyme is not present in the cell broth of C. histolyticum (Clone 004).

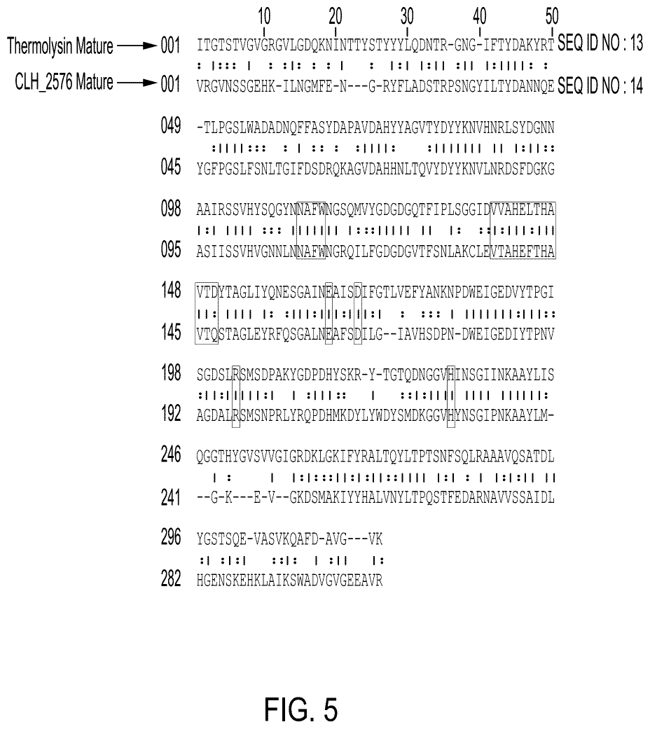

To explore the mature forms of both proteins, the comparative sequence alignment is depicted in FIG. 5. An inspection of the sequence alignment in FIG. 5 suggests that many essential amino acids have been conserved. Notably, the AHELTHA VTD sequence (SEQ ID NO: 28) of the Mature Thermolysin, beginning with Ala140 of SEQ. ID. NO. 13 has been identified as a component of the active site for thermolysin and the high homology displayed by CLH_2576 of SEQ ID. NO. 14 suggests that CLH delta toxin is a member of the thermolysin class of proteases (Kooi et al. (1996), J Med Microbiol 45: 219-225; Kooi, et al., (1997), Infect Immun 65: 472-477). Multiple residues shaded in green have been identified as essential for binding or catalysis. One notable difference between the sequences of the two molecules is the GGI region beginning with G135 in thermolysin. This stretch of amino acid residues is highly conserved in thermolysin-like proteases with no defined function assigned (Frigerio et al. (1997), Protein Eng 10: 223-230). The corresponding CLH _2576 region possesses several significant differences in this sequence. Nevertheless, the overall high degree of homology and the conservation of essential amino acid residues confirm the selection of CLH_2576 as delta toxin with predicted molecular mass of approximately 35 kDa. This assessment aligns with the information presented by Takahashi et al (1970) (BBRC 39: 1058-1064).

In summary, the putative CLH delta toxin has been identified using genome sequence analysis. However, the interrogation of this sequence suggests that the cleavage of the proprotein will not occur, rendering this molecule non-functional. Therefore, it is deduced that the 8 toxin, if expressed and secreted in the Clone 004 derivative of C. histolyticum ATCC 21000, is not functional.

### 3. Epsilon (ε) Toxin

MacLennan et al. (1962) (Bact Rev 26: 176-274) and Hatheway described the ε toxin of C. histolyticum as an oxygen-labile haemolysin serologically similar to those produced by other strains of Clostridium, such as C. tetani, C. novyi, and C. septicum. Bowen (1952) (Yale J Biol Med 25: 124-138) demonstrated that the ε toxin was expressed during the exponential phase and degraded during the stationary phase as observed for the a toxin activity, and was similarly degraded by proteinases in vitro.

An inspection of the BLAST analysis results of the C. histolyticum genome identified an ORF coding for a hemolysin that was in the same class as perfringolysin and tetanolysin, which are members of thiol-activated, pore forming proteins with affinity for cholesterol. Such proteins are part of a family of Cholesterol Dependent Cytolysins (CDC) and all exhibit distinctive protein sequences and unique structures. Over 25 CDC proteins have been identified with complete protein sequences available. The CDCs are a group of β-barrel pore-forming toxins secreted by various species of Gram positive bacteria all in the 50-60 kDa molecular weight range. The prototypical CDC is perfringolysin which serves as a model protein for all CDCs (Heuck et al. 2007, JBC 282: 22629-22637). The typical organization of a CDC includes a cleavable signal sequence to facilitate the exports to the extracellular environment as a water-soluble monomeric protein. Subsequently, the folded monomeric form binds to a target eukaryotic membrane, mediated by cholesterol binding, and then oligomerizes on the membrane surface to form arcs and ring-like structures that are responsible for the cytolysis. The CDCs are also known as thiol-activated cytolysins and were originally described as hemolysins (Billington et al., 2000).

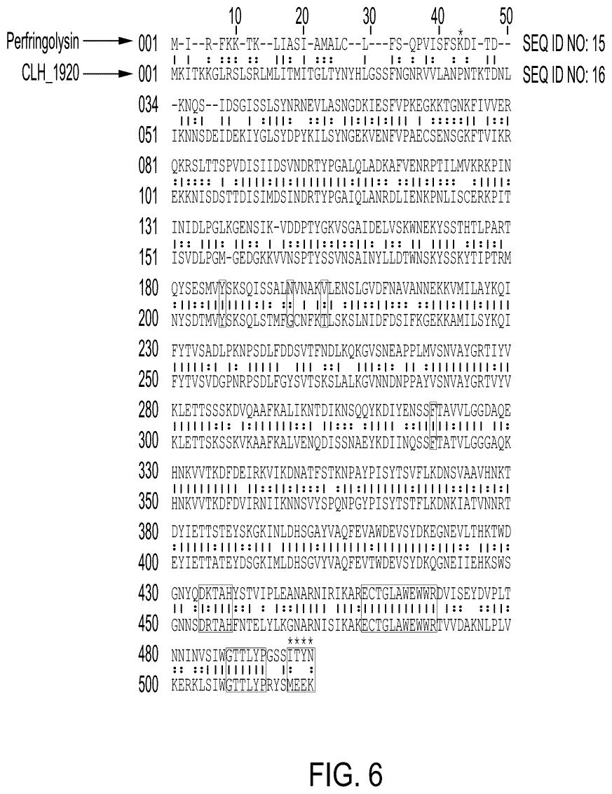

The gene sequence alignment for perfringolysin and CLH_1920, the putative epsilon toxin, is illustrated in FIG. 6. This image displays the full length (pre plus mature) protein sequence as a single unit that is theoretically transcribed as a single polypeptide. The initial 29 amino acids at the N-terminus of perfringolysin are illustrated with a blue star above Lys 29 at the site of signal peptidase cleavage. The SignalP analysis of the CLH_1920 sequence did not identify a recognizable signal peptide cleavage site and was predicted to be a non-secreted protein. There is an 84% positive homology between the perfringolysin protein sequence and the CLH_1920 putative epsilon toxin protein sequence.

The amino acid residues shaded in green denote essential amino acids that are conserved between the two proteins. Importantly, the 11 amino acid sequence ECTGLAWEWWR (SEQ ID NO: 29), beginning with glutamine 458 of SEQ. ID. NO. 15, is an essential region that is termed the undecapeptide sequence. Along with the high degree of homology within the sequence designated as the mature protein region, this undecapeptide sequence serves to identify the CLH_1920 protein as a CDC. Therefore, the CLH_1920 protein, if elaborated as a secreted protein, would be expected to have haemolytic functionality. A single region of non-homology between the two proteins is highlighted in yellow shading. Importantly, the C-terminus of CDCs has been shown to be critical for cholesterol binding (Shimada et al., 1999, JBC 274: 18536-18542). The process of hemolysis by CDCs involves two critical steps prior to pore formation: binding and membrane insertion. Shimada, et al. (1999) (JBC 274: 18536-18542) demonstrated that modest changes to the C-terminus affected the binding step. An alteration of the 3′ terminal amino acids severely reduces cholesterol binding as measured by an ELISA method. The corresponding haemolytic activity on red blood cells was coordinately reduced or eliminated depending upon the severity of the C-terminal amino acid change. An inspection of the C-terminus of the CLH_1920 sequence shows some significant differences compared to the perfringolysin sequence.

As summarized in Table 4, the haemolytic activity of the putative C. histolyticum epsilon toxin may be absent due to two features of the theoretical amino acid sequence. First, the molecule is predicted not be secreted; thus, the molecule would not be available for interaction with target cells. Second, the C-terminus of CLH_1920 protein does not possess a homologous region for cholesterol binding, which suggests that an important element associated with hemolysis may be defective.

TABLE 4
Summary of Amino Acid Sequence
Alignment Comparison for Putative CLH epsilon toxin

| Protein | | |
|---|---|---|
| Perfringolysin | CLH 1920 | |
| Region | Characteristic | Effect on Function |
| N-terminal | Missing signal | Not secreted |
| | peptidase cleavage | |
| | sequence | |
| C-terminal | Non-consensus | Lack of cholesterol |
| | | binding/no activation |

Non-clinical toxicity studies demonstrated no clinical and morphological indications of hemolysin effects in vivo. The data generated by local and IV bolus administration support the absence of haemolytic toxins such as ε toxin.

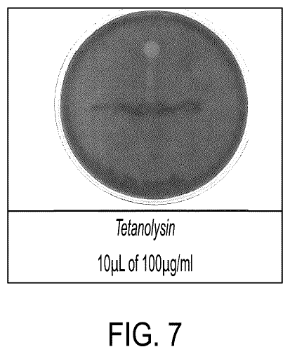

The absence of haemolytic toxins can be verified by the plating of test material on blood agar which is routinely performed at the end of each C. histolyticum Clone 004 fermentation, which also confirms the absence of foreign growth. The expression of haemolytic toxins results in the lysis of the blood cells, and thereby resulting in the formation of distinct halos around colonies producing haemolysins. The Collagenase C. histolyticum production strain does not produce halos or zones of clearance (see FIG. 2) supporting the absence of ε toxin and any other haemolytic entities in the production strain. To verify the hemolytic function of a CDC, commercially available tetanolysin was applied to Blood agar to mimic the routine plating test. The results are illustrated in FIG. 7 which shows the beta hemolytic phenotype observed when 10 μcL of a 10 μg/mL solution of tetanolysin in phosphate buffered saline is applied to the surface of Blood agar, then incubated for 24 hours at 37° C. Thus, if a functional CDC were present in the test material, the beta hemolytic phenotype should be observed.

### 4. Clostripain or Gamma (γ)-Toxin

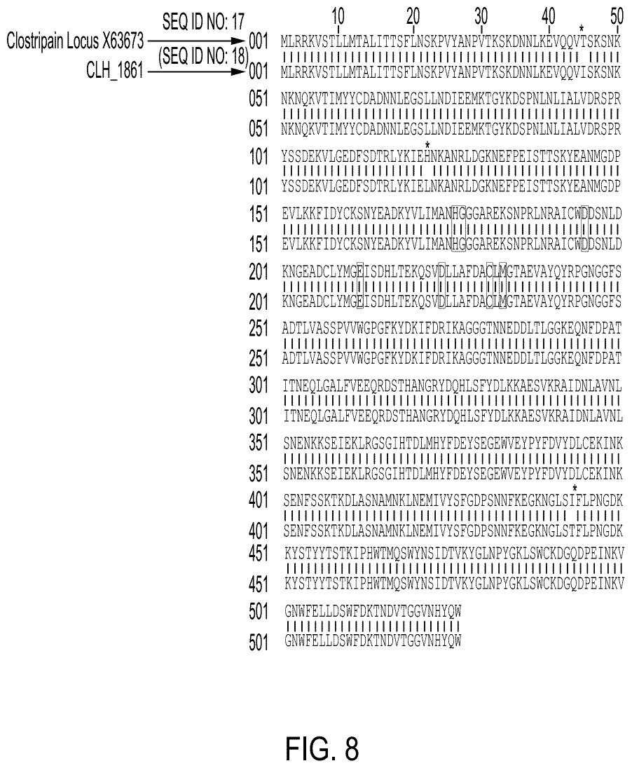

The gamma toxin of C. histolyticum has been described as clostripain, a cysteine endopeptidase (EC 3.4.22.8). Dargatz, et al. (1993) (Mol Gen Genet 240: 140-145) cloned and sequenced the C. histolyticum gene for clostripain and this information was deposited in GenBank under accession number X63673 (www.ncbi.nlm.nih.gov/nuccore/X63673.1). To understand the primary structure of the CLH_1861 gamma toxin, the protein sequence alignment from MATLAB of the model protein (C. histolyticum clostripain) was made with the CLH_1861 gamma toxin theoretical sequence. The results are presented in FIG. 8.

An inspection of FIG. 8 shows a very high degree of homology (99%) between the model clostripain and the sequence obtained from the genome analysis. In fact, there are only 3 amino acid differences, none of which are residues identified as essential for activity. Those critical amino acids identified in literature studies as essential for functionality are shown in green shading. SignalP analysis of both proteins indicated that high secretion score and the signal cleavage site depicted with a blue star (Labrou et al. (2004). Eur J Biochem 271: 983-992). Thus, one would predict that the CLH_1861 molecule would be secreted and functional. A residual clostripain analysis was conducted as part of routine release.

The clostripain analysis supports the merits of the sequence alignment approach for the C. histolyticum toxins in general. One would predict that the presence of a functional toxin gene would necessarily translate into an amino acid sequence that shared a high degree of homology with a known model protein. Further, the conservation of essential amino acid residues would also be a characteristic of a functional toxin gene.

The information obtained from the genome sequence analysis provided evidence that loci for putative alpha, delta, and epsilon toxins were present. Further analysis of the theoretical primary structure of each toxin indicated that non-functional forms of each toxin were predicted as a consequence of key defects in the amino acid sequence of each toxin. Notably, the alpha and epsilon toxins can be assigned as homologues to two classes of pore-forming, hemolytic molecules. As the end of fermentation, samples from every batch are plated onto blood agar as part of a routine purity test. The lack of halos or zones of clearance around the colonies confirm the absence of haemolytic activity in the culture and fermentation. Consequently, the absence of haemolytic halos around the end of fermentation samples demonstrate the absence of both α and ε toxins on a continuing basis.

Table 5 shows the results from the sequence analysis and predicted functionality. The results confirm why Clone 004 has functionally shown the absences of toxicity and the lack haemolytic activity.

TABLE 5
Summary-Predicted Status of
C. histolyticum Clone 4 Exosubstances

| Toxin | CLH Name | Sequence Result | Predicted Functionality |
|---|---|---|---|
| alpha | CLH_2834 & | Missing critical aa | Non functional; |
| | 2835 | residues | correlated through |
| | | | absence of haemolytic |
| | | | activity on blood agar |
| | | | plates |
| delta | CLH_2576 | Missing consensus | Non-functional |
| | | proprotein cleavage | |
| | | sequence | |
| epsilon | CLH_1920 | Signal peptidase | Not secreted, non- |
| | | cleavage site | functional correlated |
| | | defective & non- | through absence of |
| | | consensus | haemolytic activity on |
| | | cholesterol | blood agar plates |
| | | binding sequence | |
| gamma | CLH_1861 | Clostripain | Functional |

5. C. histolyticum Sequence Analysis of Beta Toxins (Collagenase I and Collagenase II)

The sequence analysis of the putative C. histolyticum beta toxin loci is presented in FIGS. 9A, 9B, 10A, and 10B. As shown in FIG. 9A and 9B, the amino acid sequence of the mature collagenase I of clone 004 (CLH_1768 and 1769; SEQ ID NO: 3) differs from the translated colG sequence (SEQ ID NO: 19) by three amino acids. FIG. 10A and 10B show that the amino acid sequence of the mature collagenase II of clone 004 (CLH 2116; SEQ ID NO: 4) differs from the translated colH sequence (SEQ ID NO: 20) by eight amino acids. Both collagenases are fully functional.

The patent and scientific literature referred to herein establishes the knowledge that is available to those with skill in the art. All United States patents and published or unpublished United States patent applications cited herein are incorporated by references. All published foreign patents and patent applications cited herein are hereby incorporated by reference. All other published references, documents, manuscripts and scientific literature cited herein are hereby incorporated by reference.

### REFERENCES

1. Nielsen et al. (2004) In J. Glasgow et al., eds., Proc. Sixth Int. Conf. on Intelligent Systems for Molecular Biology, 122-130. AAAI Press, 1998.
2. Hatheway (1990) Clin Microbiol Rev 3: 66-98.
3. Ballard et al. (1992) Infect Immun 60: 784-790.
4. Melton-Witt et al. (2006) Biochem 45: 14347-14354.
5. Gordon et al. (1997) Infect immun 65: 4130-4134.
6. Takahashi et al. (1970) BBRC 39: 1058-1064.
7. O'Donohue & Beaumont (1996) JBC 271: 26477-26481.
8. Demidyuk et al. (2008) Protein J 27: 343-354.
9. Wetmore et al. (1994) Mol Microbiol 12: 747-759.
10. O'Donohue et al. (1994) Biochem J. 300: 599-603.
11. Kooi & Sokol (1996) J Med Microbiol 45: 219-225.
12. Kooi et al. (1997) Infect Immun 65: 472-477.
13. Frigerio et al. (1997) Protein Eng 10: 223-230.
14. MacLennan (1962) Bact Rev 26: 176-274.
15. Bowen (1952) Yale J Biol Med 25: 124-138.
16. Heuck et al. (2007) JBC 282: 22629-22637.
17. Billington et al. (2000) FEMS Microbiol Lett 182: 197-205.
18. Shimada et al. (1999) JBC 274: 18536-18542
19. Dargatz et al. (1993) Mol Gen Genet 240: 140-145.
20. Labrou & Rigden (2004) Eur J Biochem 271: 983-992.

While this invention has been particularly shown and described with references to preferred embodiments thereof, it will be understood by those skilled in the art that various changes in form and details may be made therein without departing from the scope of the invention encompassed by the appended claims.

1. A method of detecting the presence of a hemolytic toxin in a collagenase composition, the method comprising:
incubating a sample of the collagenase composition with red blood cells; and
assaying the red blood cells to detect lysis,
wherein lysis of red blood cells indicates the presence of the hemolytic toxin and an absence of lysis of red blood cells indicates an absence of the hemolytic toxin.

2. The method of claim 1, wherein the incubating is performed on a blood agar substrate.

3. The method of claim 2, wherein a zone of clearance on the blood agar substrate indicates lysis of the red blood cells.

4. The method of claim 1, wherein the assaying comprises photometric analysis.

5. The method of claim 4, wherein the photometric analysis is performed at 540 nm or 414 nm.

6. The method of claim 1, wherein the hemolytic toxin comprises epsilon toxin, alpha toxin, or a combination thereof.

7. The method of claim 1, wherein the method further comprises, prior to the incubating, producing the collagenase composition via cultivation of a collagenase producing bacterium.

8. The method of claim 7, wherein the collagenase producing bacterium is a member of the genus Actinobacillus, Actinomadura, Bacillus, Bacteriodes, Bifidobacterium, Brucella, Capnocytophaga, Clostridium, Enterococcus, Escherichia, Eubacterium, Flavobacterium, Fusobacterium, Peptococcus, Peptostreptococcus, Porphyromonas, Prevotella, Proteus, Pseudomonas, Serratia, Staphylococcus, Streptomyces, Streptococcus, Treponema, or Vibrio.

9. The method of claim 8, wherein the collagenase producing bacterium is a recombinant bacterium.

10. The method of claim 9, wherein the recombinant bacterium is E. coli.

11. The method of claim 1, wherein the collagenase is produced by recombinant techniques.
